(19) United States
(12) Patent Application Publication
(10) Pub. No.: US 2023/0000372 A1
(43) Pub. Date: Jan. 5, 2023

(54) CUFF STRUCTURE AND BLOOD PRESSURE MEASUREMENT DEVICE

(71) Applicant: OMRON HEALTHCARE Co., Ltd., Kyoto (JP)

(72) Inventors: Masaki HARADA, Kyoto (JP); Yoshihide TOKKO, Kyoto (JP)

(21) Appl. No.: 17/930,290

(22) Filed: Sep. 7, 2022

Related U.S. Application Data
(63) Continuation of application No. PCT/JP2021/005329, filed on Feb. 12, 2021.

(30) Foreign Application Priority Data
Mar. 12, 2020 (JP) 2020-043243

(51) Int. Cl. A61B 5/022; A61B 5/00

(52) U.S. Cl. CPC A61B 5/02233; A61B 5/681

(57) ABSTRACT

A cuff structure is provided in a curler of a blood pressure measurement device that includes a device body and the curler provided on the device body. The curler curves following along a circumferential direction of a wrist. The cuff structure includes a sensing cuff provided on a side of an inner circumferential surface of the curler and contacting a region where an artery is present in the wrist and a pressing cuff having a portion provided between the curler and the sensing cuff, having a portion of another portion of the cuff disposed in a region where the sensing cuff is present in an outer circumferential surface of the curler with the blood pressure measurement device attached to the wrist, and inflating to press the sensing cuff to the wrist.

### CROSS-REFERENCE TO RELATED APPLICATIONS

This application is the U.S. national stage application filed pursuant to 35 U.S.C. 365(c) and 120 as a continuation of International Patent Application No. PCT/JP2021/005329, filed Feb. 12, 2021, which application claims priority to Japanese Patent Application No. 2020-043243, filed Mar. 12, 2020, which applications are incorporated herein by reference in their entireties.

### TECHNICAL FIELD

The present invention relates to a cuff structure used in a blood pressure measurement device and the blood pressure measurement device.

### BACKGROUND ART

In recent years, blood pressure measurement devices used for measuring blood pressure are being used as means to check health status at home, as well as in medical facilities. A blood pressure measurement device detects vibration of the artery wall to measure blood pressure by, for example, inflating and contracting a cuff wrapped around the upper arm or the wrist of a living body and detecting the pressure of the cuff using a pressure sensor.

As such a blood pressure measurement device, a so-called integrated type in which a cuff is integrated with a device body feeding a fluid to the cuff is known. Moreover, as the above-described integrated blood pressure measurement device, wearable devices attached to a wrist have been considered (for example, see Patent Document 1).

### CITATION LIST—PATENT LITERATURE

Patent Document 1: JP 2019-118418 A

### SUMMARY OF INVENTION

### Technical Problem

The blood pressure measurement device described above is required to allow a sensing cuff to be in close contact with a region where an artery is present in a wrist.

Accordingly, an object of the present invention is to provide a cuff structure that allows a sensing cuff to be in close contact with a region where an artery is present in a wrist and a blood pressure measurement device.

### Solution to Problem

According to one aspect, there is provided a cuff structure provided in a curler of a blood pressure measurement device that includes a device body and the curler provided in the device body. The curler curves following along a circumferential direction of a wrist. The cuff structure includes a sensing cuff and a cuff. The sensing cuff is provided on a side of an inner circumferential surface of the curler. The sensing cuff contacts a region where an artery is present in the wrist. The cuff has a portion provided between the curler and the sensing cuff. A portion of another portion of the cuff is disposed in a region where the sensing cuff is present in an outer circumferential surface of the curler with the blood pressure measurement device attached to the wrist. The cuff inflates to press the sensing cuff against the wrist.

The sensing cuff and the cuff inflate through feed of a fluid and include bag-like structures, such as air bags.

According to this aspect, the sensing cuff is pressed by the cuff disposed between the sensing cuff and the curler and the cuff disposed on the outer circumferential surface of the curler to press the sensing cuff against the wrist. As a result, a pressing force that presses the sensing cuff against the region where the artery is present in the wrist can be increased, and thus the sensing cuff can be in close contact with the wrist.

In the cuff structure according to the one aspect described above, the following cuff structure is provided. The cuff has a length such that the portion of the other portion is disposed in the region where the sensing cuff is present in the outer circumferential surface of the curler when the cuff is attached to the wrist having any circumference from an assumed shortest circumference to an assumed longest circumference of the wrist.

According to this aspect, the sensing cuff can be in close contact with the region where the artery is present in the wrist having any circumference from the assumed shortest circumference to longest circumference of the wrist.

In the cuff structure according to the one aspect described above, the following cuff structure is provided. Both ends of the curler are spaced apart. The cuff is configured to have a shape extending from the portion to the portion of the other portion.

According to this aspect, an increase in the number of components of the cuff structure can be prevented.

In the cuff structure according to the one aspect described above, the following cuff structure is provided. The cuff includes a first cuff and a second cuff. The first cuff is provided between the curler and the sensing cuff. The second cuff is provided on a back of a hand of the wrist of the curler. A portion of the second cuff is disposed in the region where the sensing cuff is present in the outer circumferential surface of the curler with the blood pressure measurement device attached to the wrist.

According to this aspect, in a configuration in which the first cuff and the second cuff are provided in addition to the sensing cuff as well, the sensing cuff is pressed by the first cuff disposed between the sensing cuff and the curler and the second cuff disposed on the outer circumferential surface of the curler, and thus the pressing force of pressing the sensing cuff against the wrist can be improved, so the sensing cuff can be in close contact with the wrist.

According to an aspect, there is provided a blood pressure measurement device that includes a device body, a curler, a sensing cuff, and a cuff. The curler is provided in the device body and curving following along a circumferential direction of a wrist. The sensing cuff is provided on a side of an inner circumferential surface of the curler. The sensing cuff contacts a region where an artery is present in the wrist. The cuff has a portion provided between the curler and the sensing cuff. A portion of another portion of the cuff is disposed in a region where the sensing cuff is present in an outer circumferential surface of the curler with the curler, the sensing cuff, and the cuff attached to the wrist. The cuff inflates to press the sensing cuff against the wrist.

Here, the state where the curler, the sensing cuff, and the cuff are attached to the wrist is a state where the blood pressure measurement device is attached to the wrist.

According to this aspect, the sensing cuff is pressed by the cuff disposed between the sensing cuff and the curler and the cuff disposed on the outer circumferential surface of the curler to press the sensing cuff against the wrist. As a result, a pressing force that presses the sensing cuff against the region where the artery is present in the wrist can be increased, and thus the sensing cuff can be in close contact with the wrist.

In the blood pressure measurement device according to the one aspect described above, there is provided the following blood pressure measurement device. The cuff has a length such that the portion of the other portion is disposed in the region where the sensing cuff is present in the outer circumferential surface of the curler when the curler, the sensing cuff, and the cuff are attached to the wrist having any circumference from an assumed shortest circumference to an assumed longest circumference of the wrist.

According to this aspect, the sensing cuff can be in close contact with the region where the artery is present in the wrist having any circumference from the assumed shortest circumference to longest circumference of the wrist.

In the blood pressure measurement device according to the one aspect described above, there is provided the following blood pressure measurement device. Both ends of the curler are spaced apart. The cuff is configured to have a shape extending from the portion to the portion of the other portion.

According to this aspect, an increase in the number of components of the blood pressure measurement device can be prevented.

In the blood pressure measurement device according to the one aspect described above, there is provided the following blood pressure measurement device. The cuff includes a first cuff and a second cuff. The first cuff is provided between the curler and the sensing cuff. The second cuff is provided on a back of a hand of the wrist of the curler. A portion of the second cuff is disposed in the region where the sensing cuff is present in the outer circumferential surface of the curler with the curler, the sensing cuff, the first cuff, and the second cuff attached to the wrist.

According to this aspect, in a configuration in which the first cuff and the second cuff are provided in addition to the sensing cuff as well, the sensing cuff is pressed by the first cuff disposed between the sensing cuff and the curler and the second cuff disposed on the outer circumferential surface of the curler, and thus the pressing force of pressing the sensing cuff against the wrist can be improved, so the sensing cuff can be in close contact with the wrist.

In the blood pressure measurement device according to the one aspect described above, the following blood pressure measurement device is provided. The curler has a first end and a second end spaced apart. The first end overlaps with a portion of another portion of the curler.

According to this aspect, the outer circumferential surface of the cuff is supported by the curler. Furthermore, since the cuff is supported by the curler, a direction of inflation of the cuff by the inflation of the cuff can be a direction in which the sensing cuff is pressed against the wrist. Accordingly, inflation of the cuff can be efficiently used as the pressing force of pressing the sensing cuff to the wrist.

### Advantageous Effects of Invention

The present invention can provide a cuff structure that allows a sensing cuff to be in close contact with a region where an artery is present in a wrist and a blood pressure measurement device.

### BRIEF DESCRIPTION OF DRAWINGS

Various embodiments are disclosed, by way of example only, with reference to accompanying schematic drawings in which corresponding reference symbols indicate corresponding parts, in which:

FIG. 1 is a perspective view illustrating a configuration of a blood pressure measurement device according to a first embodiment of the present invention;

FIG. 2 is an explanatory diagram illustrating a state where the blood pressure measurement device is attached to a wrist;

FIG. 3 is a cross-sectional view illustrating a configuration of a curler and a cuff structure of the blood pressure measurement device;

FIG. 4 is a cross-sectional view illustrating the configuration of the curler and the cuff structure;

FIG. 5 is a plan view illustrating the configuration of the cuff structure;

FIG. 6 is a plan view illustrating the configuration of the cuff structure;

FIG. 7 is a cross-sectional view illustrating a configuration of a sensing cuff of the cuff structure;

FIG. 8 is a perspective view illustrating an example in which the blood pressure measurement device is attached to the wrist;

FIG. 9 is a perspective view illustrating an example in which the blood pressure measurement device is attached to the wrist;

FIG. 10 is a perspective view illustrating an example in which the blood pressure measurement device is attached to the wrist;

FIG. 11 is a cross-sectional view schematically illustrating a state where the blood pressure measurement device is attached to the wrist;

FIG. 12 is an explanatory diagram illustrating a state where the blood pressure measurement device is attached to the wrist;

FIG. 13 is a side view illustrating a configuration of a blood pressure measurement device according to a second embodiment of the present invention;

FIG. 14 is an explanatory diagram illustrating a state where the blood pressure measurement device is attached to the wrist;

FIG. 15 is a cross-sectional view illustrating a configuration of a cuff structure and a curler of the blood pressure measurement device;

FIG. 16 is a plan view illustrating the configuration of the cuff structure;

FIG. 17 is a plan view illustrating the configuration of the cuff structure;

FIG. 18 is an explanatory diagram illustrating a state where the blood pressure measurement device is attached to the wrist;

FIG. 19 is an explanatory view illustrating a state where a blood pressure measurement device according to a third embodiment of the present invention is attached to the wrist;

FIG. 20 is a plan view illustrating a configuration of a cuff structure of the blood pressure measurement device;

FIG. 21 is a plan view illustrating the configuration of the cuff structure;

FIG. 22 is an explanatory view illustrating a state where a blood pressure measurement device according to a fourth embodiment of the present invention is attached to the wrist;

FIG. 23 is a plan view illustrating a configuration of a cuff structure of the blood pressure measurement device;

FIG. 24 is an explanatory diagram illustrating a state where the blood pressure measurement device is attached to the wrist;

FIG. 25 is an explanatory diagram illustrating a state where a blood pressure measurement device according to a modified example of the first to fourth embodiments is attached to the wrist;

FIG. 26 is an explanatory diagram illustrating a state where the blood pressure measurement device is attached to the wrist;

FIG. 27 is an explanatory diagram illustrating a state where the blood pressure measurement device according to a modified example of the first to fourth embodiments is attached to the wrist;

FIG. 28 is an explanatory diagram illustrating a state where the blood pressure measurement device according to a modified example of the first to fourth embodiments is attached to the wrist; and,

FIG. 29 is an explanatory diagram illustrating a state where the blood pressure measurement device is attached to the wrist.

### DESCRIPTION OF EMBODIMENTS

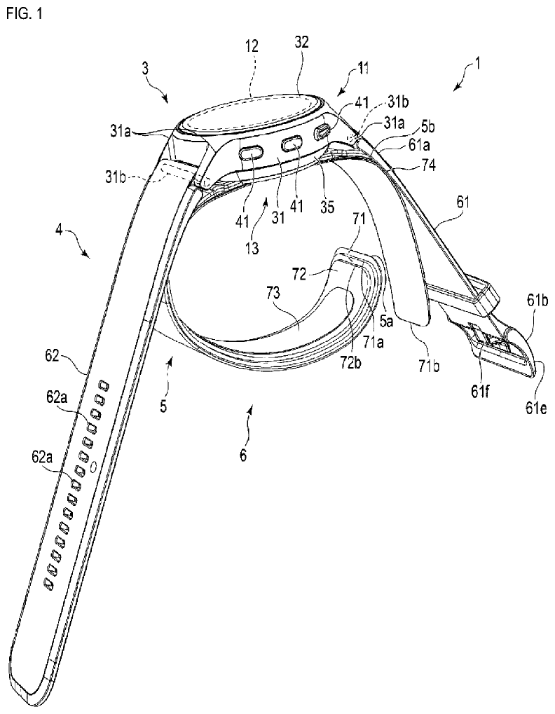

An example of a blood pressure measurement device 1 according to a first embodiment of the present invention will be described below with reference to FIGS. 1 to 12.

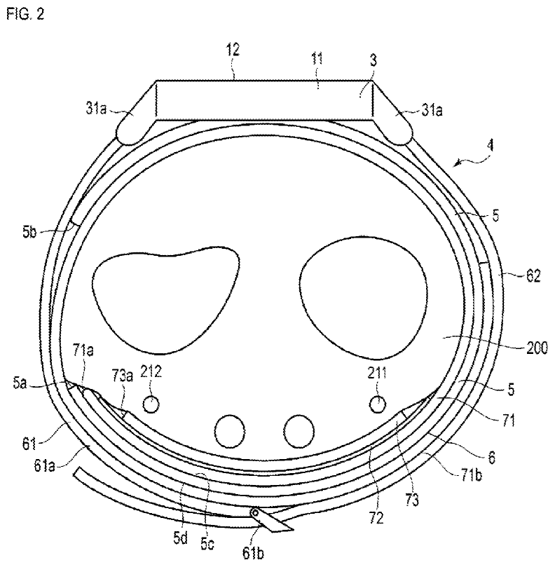

FIG. 1 is a perspective view illustrating a configuration of the blood pressure measurement device 1 according to a first embodiment of the present invention. FIG. 2 is an explanatory diagram illustrating a state where the blood pressure measurement device 1 is attached to a wrist 200. FIG. 2 illustrates an example of a state where the blood pressure measurement device 1 is attached to the wrist 200 having the assumed shortest circumference as the circumference of the wrist 200 among the wrists 200 of a plurality of users set as targets using the blood pressure measurement device 1. A cuff structure 6 illustrated in FIG. 2 is in an inflated state.

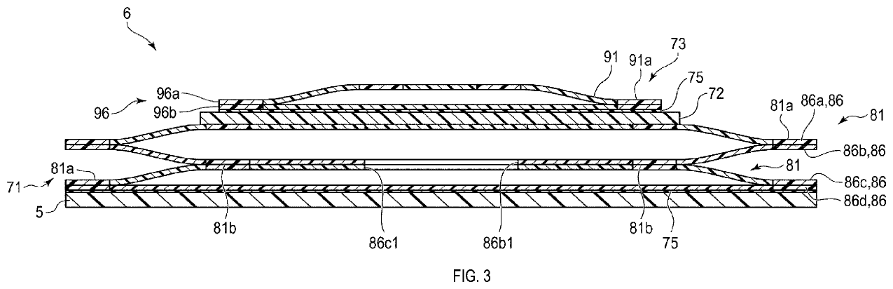

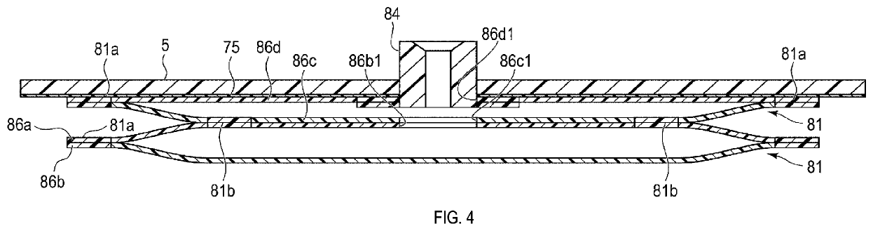

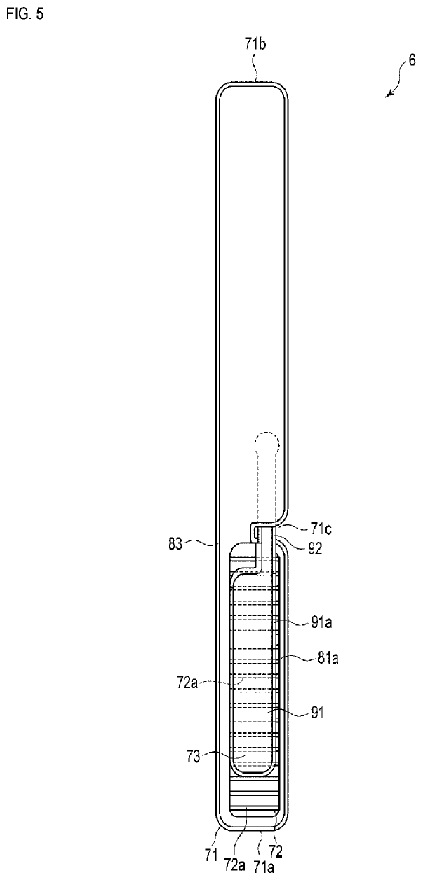

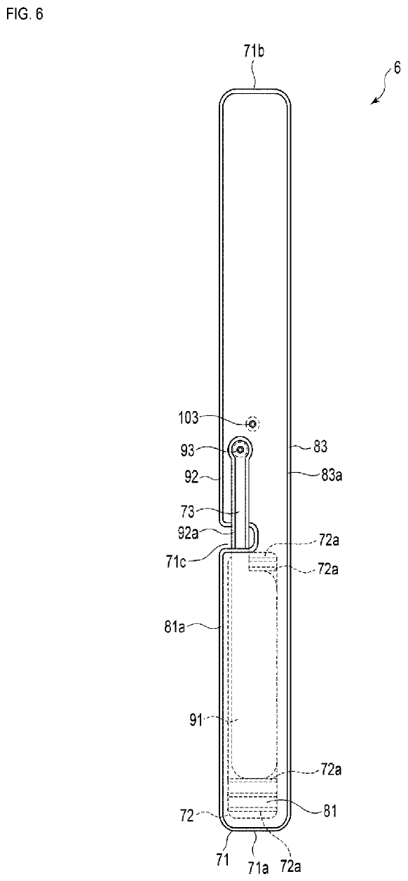

FIG. 3 is a cross-sectional view illustrating the configuration of a curler 5 and the cuff structure 6 of the blood pressure measurement device 1. FIG. 4 is a cross-sectional view illustrating the configuration of the curler 5 and the cuff structure 6 of the blood pressure measurement device 1. FIG. 5 is a plan view illustrating a surface of the cuff structure 6 of the blood pressure measurement device 1 fixed to an inner circumferential surface of the curler 5. FIG. 6 is a plan view illustrating the surface of the configuration of the cuff structure 6 fixed to an inner circumferential surface 5c of the curler 5.

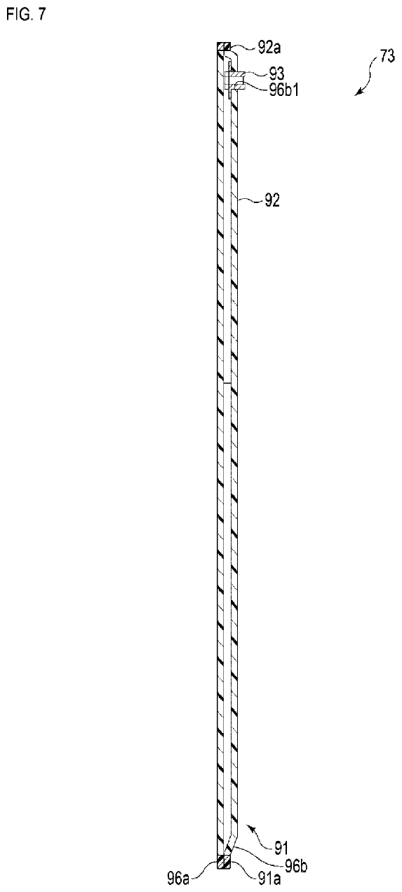

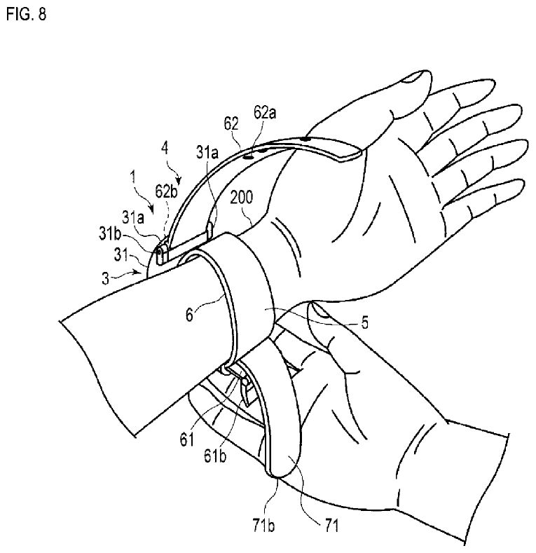

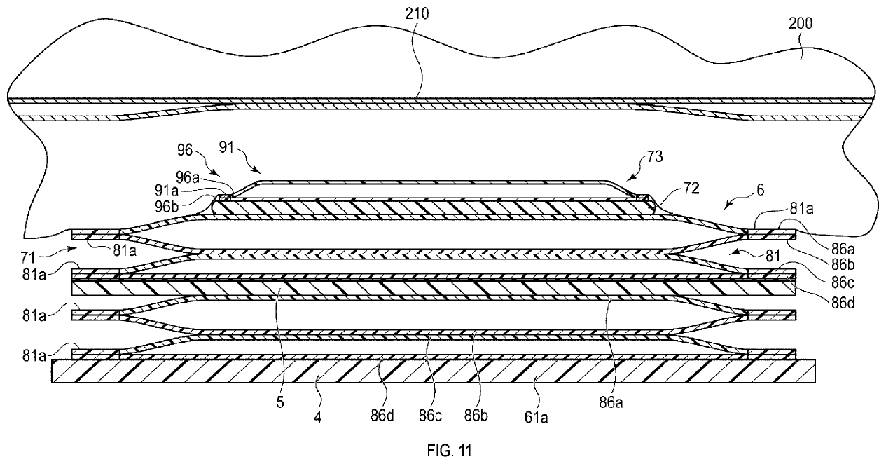

FIG. 7 is a cross-sectional view illustrating a configuration of a pressing cuff 71 of the cuff structure 6. FIGS. 8 to 10 are perspective views illustrating an example in which the blood pressure measurement device 1 is attached to the wrist 200. FIG. 11 is a cross-sectional view schematically illustrating a state where the blood pressure measurement device 1 is attached to the wrist 200.

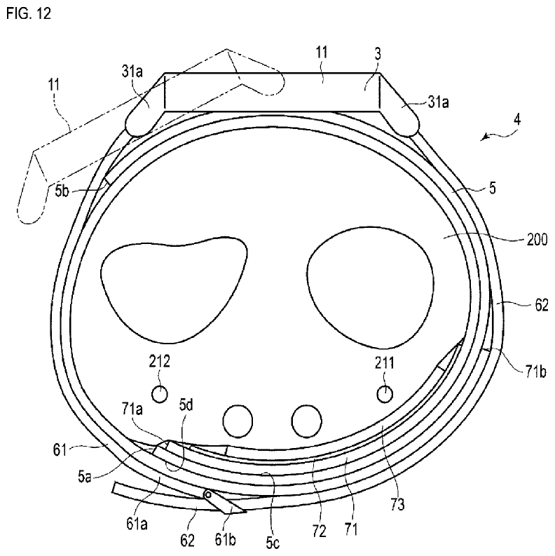

FIG. 12 is an explanatory diagram illustrating a state where the blood pressure measurement device 1 is attached to the wrist 200. FIG. 12 illustrates an example of a state where the blood pressure measurement device 1 is attached to the wrist 200 having the assumed longest circumference as the circumference of the wrist 200 among the wrists 200 of the plurality of users assumed as targets using the blood pressure measurement device 1. The cuff structure 6 illustrated in FIG. 12 is in an inflated state.

As illustrated in FIGS. 1 and 2, the blood pressure measurement device 1 includes a device body 3, a belt 4 that fixes the device body 3 to the wrist 200, the curler 5 disposed between the belt 4 and the wrist 200, and the cuff structure 6.

The device body 3 includes, for example, a case 11, a display unit 12, and an operation unit 13. The device body 3 includes a pump to inflate the cuff structure 6, a flow path portion that fluidly connects the pump and the cuff structure 6, and a control substrate in the case 11.

The case 11 includes an outer case 31 and a windshield 32 that covers an opening of the outer case 31 on the side opposite to the wrist 200 side.

The outer case 31 is formed in a cylindrical shape. The outer case 31 includes pairs of lugs 31a provided at respective symmetrical positions in the circumferential direction of an outer circumferential surface, and spring rods 31b each provided between each of the two pairs of lugs 31a.

The windshield 32 is, for example, a circular glass plate.

The display unit 12 is disposed directly below the windshield 32. As illustrated in FIG. 1, the display unit 12 is electrically connected to the control substrate. The display unit 12 is, for example, a liquid crystal display or an organic electroluminescence display. The display unit 12 displays various types of information including the date and time and measurement results of blood pressure values such as the systolic blood pressure and diastolic blood pressure, heart rate, and the like.

The operation unit 13 is configured to be capable of receiving an instruction input from a user. For example, as illustrated in FIG. 1, the operation unit 13 includes a plurality of buttons 41 provided on the case 11 and a sensor that detects operations of the buttons 41. As the plurality of buttons 41, for example, three buttons are provided.

The belt 4 includes a first belt 61 provided on the first pair of lugs 31a and a first spring rod 31b and a second belt 62 provided on the second pair of lugs 31a and a second spring rod 31b. The belt 4 is wrapped around the wrist 200 with the curler 5 in between.

The first belt 61 is referred to as a so-called parent and is configured like a band capable of being joined to the second belt 62. The first belt 61 includes a belt portion 61a and a buckle 61b. The belt portion 61a is configured like a band. The belt portion 61a is formed of an elastically deformable resin material.

A first end of the belt portion 61a is supported by a first spring rod 31b. The buckle 61b is provided at the second end of the belt portion 61a. The buckle 61b includes a frame body 61e in a rectangular frame shape and a prong 61f rotatably attached to the frame body 61e.

The second belt 62 is referred to as a so-called blade tip, and is configured in a band-like shape having a width at which the second belt 62 can be inserted into the frame body 61e. The second belt 62 is formed of an elastically deformable resin material. In addition, the second belt 62 includes a plurality of small holes 62a into which the prong 61f is inserted. A first end of the second belt 62 is supported by the second spring rod 31b.

The second belt 62 is inserted into the frame body 61e and the prong 61f is inserted into the small hole 62a, and thus the first belt 61 and the second belt 62 are integrally connected together, and the belt 4 thus configured together with the outer case 31, comes to have an annular shape following along the circumferential direction of the wrist 200. By shaping the belt 4 in the annular shape following along the circumferential direction of the wrist 200, the curler 5 is pressed and elastically deformed to follow along the circumferential direction of the wrist 200 of a wearer of the blood pressure measurement device 1.

As illustrated in FIGS. 1 and 2, the curler 5 is configured in a band-like shape that curves in such a manner as to follow along the circumferential direction of the wrist 200. The curler 5 is formed with a first end 5a and a second end 5b spaced apart from one another. For example, the outer circumferential surface on the second end 5b side of the curler 5 is fixed to the device body 3. The first end 5a is a first end on a hand palm side of the wrist 200. The second end 5b is a first end on a hand back side of the wrist 200. The curler 5 is formed of a resin material, for example.

The length of the curler 5 from the device body 3 to the second end 5b is formed to be shorter than the length from the device body 3 to the first end 5a. In the curler 5, the shorter side from the device body 3 to the second end 5b is disposed on the hand back side of the wrist 200. The longer side from the device body 3 to the first end 5a of the curler 5 passes through one lateral side from the hand back side of the wrist 200 and extends to the hand palm side of the wrist 200.

The curler 5 with such a configuration is fixed to the outer case 31 with the first end 5a and the second end 5b orientated to face the first belt 61 of the belt 4.

The curler 5 has a hardness appropriate to provide flexibility and shape retainability. Here, “flexibility” refers to deformation of the shape of the curler 5 in a radial direction at the time of application of an external force of the belt 4 to the curler 5. For example, “flexibility” refers to deformation of the shape of the curler 5 in a side view in which the curler 5 approaches the wrist 200, is along the shape of the wrist 200, or follows the shape of the wrist 200 when the curler 5 is pressed by the belt 4. Furthermore, “shape retainability” refers to the ability of the curler 5 to maintain a pre-imparted shape when no external force is applied to the curler 5. For example, “shape retainability” refers to, in the present embodiment, the ability of the curler 5 to maintain the shape in a shape curving along the circumferential direction of the wrist 200.

In the curler 5, the cuff structure 6 is disposed on the inner circumferential surface. The curler 5 holds a portion of the cuff structure 6 along the shape of the inner circumferential surface 5c of the curler 5. For example, the curler 5 holds the cuff structure 6 by fixing the cuff structure 6 by a joining layer 75 provided between the curler 5 and the cuff structure 6. In the present embodiment, the joining layer 75 is adhesive or double-sided tape.

In the present embodiment, as illustrated in FIGS. 1 to 7, the cuff structure 6 includes a pressing cuff (cuff) 71, a back plate 72, and a sensing cuff 73. The pressing cuff 71 is an example of the cuff disposed between the sensing cuff 73 and the curler 5 and in a region where the sensing cuff 73 is present in an outer circumferential surface 5d of the curler 5. The cuff structure 6 is provided with the joining layer 75 for joining respective mutual components and the curler 5 and the pressing cuff 71 together.

In the cuff structure 6, the pressing cuff 71, the back plate 72, and the sensing cuff 73 are stacked and are disposed on the curler 5. As a specific example, in the cuff structure 6, as illustrated in FIGS. 1 and 2, the pressing cuff 71 is fixed to the inner circumferential surface 5c of the curler 5. Furthermore, the back plate 72 is fixed to the inner circumferential surface on the hand palm side of the wrist 200 of the pressing cuff 71 from the inner circumferential surface of the pressing cuff 71 to the wrist 200 side. Furthermore, the sensing cuff 73 is fixed to the inner circumferential surface on the hand palm side of the back plate 72. Each of the members of the cuff structure 6 is fixed to an adjacent member in a stacking direction by the joining layer 75.

The pressing cuff 71 is fluidly connected to the pump via the flow path portion. The pressing cuff 71 is configured in a band-like shape extending in one direction. A portion of the pressing cuff 71 is fixed to the inner circumferential surface of the curler 5 by the joining layer 75.

In addition, the pressing cuff 71 is set to have a length such that a portion of the pressing cuff 71 is located in the region where the sensing cuff 73 is present in the outer circumferential surface 5d of the curler 5 when the blood pressure measurement device 1 is attached to the wrist 200 having any circumference from the assumed shortest circumference to the assumed longest circumference of the wrist 200.

Thus, the pressing cuff 71 is configured in a shape that protrudes in the circumferential direction of the curler 5 beyond the second end 5b of the curler 5. A portion of the pressing cuff 71 protruding in the circumferential direction of the curler 5 beyond the second end 5b of the curler 5 is disposed at least in the region where at least the sensing cuff 73 is present in the outer circumferential surface 5d of the curler 5 with the blood pressure measurement device 1 attached to the wrist 200.

In other words, the length of the portion of the pressing cuff 71 protruding beyond the second end 5b of the curler 5 is set to be the length such that the pressing cuff 71 is located in the region where at least the sensing cuff 73 is present in the outer circumferential surface 5d of the curler 5 when the blood pressure measurement device 1 is attached to the wrist 200 having any circumference from the assumed shortest circumference to the assumed longest circumference of the wrist 200.

Here, as illustrated in FIG. 2, the wrist 200 having the assumed shortest circumference of the wrist 200 is the wrist 200 having the shortest circumference of the wrist 200 among the wrists 200 of the plurality of users set as targets using the blood pressure measurement device 1. As illustrated in FIG. 12, the wrist 200 having the assumed longest circumference of the wrist 200 is the wrist 200 of the user having the longest circumference of the wrist 200 among the wrists 200 of the plurality of users assumed as targets using the blood pressure measurement device 1.

Furthermore, the length of the portion of the pressing cuff 71 protruding from the second end 5b of the curler 5 is set to have a length not in contact with the device body 3 in a state where the blood pressure measurement device 1 is attached to the wrist 200 having the assumed shortest circumference of the wrist 200.

A first end 71a of the pressing cuff 71 is disposed, for example, in the vicinity of the first end 5a located on the hand palm side of the wrist 200 of the inner circumferential surface 5c of the curler 5.

Additionally, as illustrated in FIGS. 5 and 6, the pressing cuff 71 includes an insertion portion 71c that allows disposing a portion of the sensing cuff 73 in, for example, a middle abdomen in the longitudinal direction of the pressing cuff 71. The insertion portion 71c is formed, for example, in a shape in which a portion of the edge portion along the longitudinal direction of the pressing cuff 71 is recessed in the lateral direction of the pressing cuff 71. The insertion portion 71c is configured to allow the sensing cuff 73 to pass from the inner circumferential surface side of the pressing cuff 71 to the curler 5 side.

As illustrated in FIGS. 3 and 4, for example, the pressing cuff 71 includes a plurality of air bags 81 and a connection portion 84 provided on the air bag 81 facing the curler 5. The plurality of air bags 81 are, for example, the two air bags 81. The pressing cuff 71 with such a configuration is configured by integrally welding a plurality of sheet members 86 together. The connection portion 84 is connected to the flow path portion. The connection portion 84 is connected to the flow path portion, and thus the pressing cuff 71 is fluidly connected to the pump.

Here, the air bags 81 are bag-like structures, and in the present embodiment, the blood pressure measurement device 1 is configured to use air with the pump, and thus the present embodiment will be described using the air bags. However, in a case where a fluid other than air is used, the bag-like structures may be fluid bags that are inflated by the fluid. The plurality of air bags 81 are stacked and are in fluid communication with one another in the stacking direction.

Each of the air bags 81 is formed in a rectangular bag-like shape that is long in one direction. Additionally, the air bags 81 are set so that the width in the lateral direction is the same as the width in the lateral direction of the curler 5. The air bag 81 is constituted by, for example, combining two sheet members 86 and, as illustrated in FIGS. 3 to 6, welding weld portions 81a using heat into a rectangular frame shape long in one direction.

In addition, the two-layer air bags 81 are constituted by integrally combining the two air bags 81 by welding using heat, or by welding the facing sheet members 86 of the adjacent air bags 81, and after that welding the facing sheet members 86 constituting the air bags 81 to the respective facing sheet members 86.

As a specific example, the two-layer air bags 81 fluidly communicate with openings provided in the sheet members 86 facing one another. In addition, in the two-layer air bags 81, by bridge welding the facing sheet members 86 together in a quadrilateral frame shape smaller than the weld portion 81a located on the outer peripheral edge and surrounding the plurality of openings with a bridge weld portion 81b, and thus the adjacent air bags 81 are integrally formed. The two-layer air bags 81 fluidly communicate at the insides of the bridge weld portions 81b. Here, bridge in bridge welding and the bridge weld portion 81b means integrally joining adjacent air bags 81.

The connection portion 84 is, for example, a nipple. The connection portion 84 is connected to the flow path portion of the device body 3. The connection portion 84 is provided, for example, in a portion facing the device body 3 of the air bag 81, which is disposed adjacent to the curler 5. The leading end of the connection portion 84 is exposed from a sheet member 86 facing the curler 5, among the two sheet members 86 forming the air bag 81. The connection portion 84 is connected to the flow path portion.

As a specific example, as illustrated in FIGS. 3 and 4, the pressing cuff 71 includes a first sheet member 86a, a second sheet member 86b, a third sheet member 86c, and a fourth sheet member 86d in this order from the wrist 200 side. The second sheet member 86b constitutes the first-layer air bag 81 along with the first sheet member 86a, the third sheet member 86c is integrally joined to the second sheet member 86b, and the fourth sheet member 86d constitutes the second-layer air bag 81 and a flow path body 83 along with the third sheet member 86c. Note that the pressing cuff 71 is integrally constituted by joining the adjacent sheet members 86 by welding using heat.

The first sheet member 86a and the second sheet member 86b are configured in a similar rectangular shape to the air bags 81, and peripheral edge portions of the four sides are welded to constitute the air bags 81. The second sheet member 86b and the third sheet member 86c are disposed facing one another, and include a plurality of openings 86b1 and 86c1, respectively, that cause the two air bags 81 to fluidly communicate with one another. Additionally, the second sheet member 86b and the third sheet member 86c are integrally joined by the peripheral region of the plurality of openings 86b1 and 86c1 being welded using heat in a quadrilateral frame shape smaller than the welded four sides of the air bags 81.

The third sheet member 86c is constituted in a shape that allows constituting the air bag 81, for example. The fourth sheet member 86d is constituted in a shape that allows constituting the air bag 81, for example. Furthermore, the fourth sheet member 86d includes a hole portion 86d1 into which the leading end of the connection portion 84 can be inserted, for example.

The third sheet member 86c and the fourth sheet member 86d are disposed facing one another, thermally welded along the peripheral edge shape of the air bag 81, and cut into predetermined shapes, thereby constituting the air bag 81.

In the fourth sheet member 86d, the connection portion 84 is disposed in the hole portion 86d1, and a periphery of the hole portion 86d1 is thermally welded to the connection portion 84. Furthermore, the fourth sheet member 86d is joined to the inner circumferential surface 5c of the curler 5 via the joining layer 75, and the third sheet member 86c is joined to the curler 5 via the joining layer 75.

As illustrated in FIGS. 1 and 2, the back plate 72 is formed in the plate shape long in one direction. A first end 72b in the longitudinal direction of the back plate 72 is disposed in the vicinity of the first end 71a of the pressing cuff 71, for example.

As illustrated in FIG. 3, the back plate 72 is stuck to the outer surface of the first sheet member 86a of the pressing cuff 71 by the joining layer 75. The back plate 72 has shape followability. Here, “shape followability” refers to a function in which the back plate 72 can be deformed in such a manner as to follow the shape of a contacted portion of the wrist 200 to be disposed. This contacted portion of the wrist 200 refers to a region of the wrist 200 that faces the back plate 72. This contact includes both direct contact and indirect contact via the sensing cuff 73.

For example, as illustrated in FIGS. 5 and 6, the back plate 72 includes a plurality of grooves 72a formed in both main surfaces of the back plate 72 and extending in a direction orthogonal to the longitudinal direction. The plurality of grooves 72a are provided in the respective both main surfaces of the back plate 72. The plurality of grooves 72a provided in one of the main surfaces face the corresponding grooves 72a provided in the other main surface in the thickness direction of the back plate 72. Additionally, the plurality of grooves 72a are disposed at equal intervals in the longitudinal direction of the back plate 72.

In the back plate 72, portions including the plurality of grooves 72a are thinner than portions including no grooves 72a and thus the portions including the plurality of grooves 72a are easily deformed. Accordingly, the back plate 72 is deformed in such a manner as to follow to the shape of the wrist 200, and has shape followability of extending in the circumferential direction of the wrist 200. The back plate 72 is formed such that the length of the back plate 72 is sufficient to cover the hand palm side of the wrist 200. The back plate 72 transfers the pressing force from the pressing cuff 71 to the back plate 72 side main surface of the sensing cuff 73 in a state in which the back plate 72 is extending along the shape of the wrist 200.

The sensing cuff 73 is fluidly connected to the pump via the flow path portion. The sensing cuff 73 is fixed to the main surface of the back plate 72 on the wrist 200 side by the joining layer 75. As illustrated in FIGS. 2 and 12, the length in the longitudinal direction of the sensing cuff 73 is set to have the length in contact with the region where at least one of a radial artery 211 and an ulnar artery 212, which are arteries 210 of the wrist 200, is present in the wrist 200 having any circumference from the assumed shortest circumference to the assumed longest circumference of the wrist 200.

The sensing cuff 73 is, for example, formed in the same shape as that of the back plate 72 or a shape that is smaller than that of the back plate 72, in the longitudinal direction and the width direction of the back plate 72. The sensing cuff 73 is inflated to compress a hand palm side region of the wrist 200 in which the artery 210 resides. The sensing cuff 73 is pressed by the inflated pressing cuff 71 toward the wrist 200 side with the back plate 72 in between.

The length along the longitudinal direction of the sensing cuff 73 is set to, for example, as illustrated in FIG. 2, the length at which the sensing cuff 73 contacts the region where the radial artery 211 and the ulnar artery 212 are present in the wrist 200 having the shortest circumference, and as illustrated in FIG. 12, the length at which the sensing cuff 73 contacts the region where the radial artery 211 or the ulnar artery 212 is present in the wrist 200 having the longest circumference. Specifically, the sensing cuff 73 is set to have the length substantially the same as the length from the radial artery 211 to the ulnar artery 212 in the circumferential direction of the wrist 200 having the shortest circumference.

As illustrated in FIGS. 5 to 7, the sensing cuff 73 includes, for example, one air bag 91, a flow path body 92 that communicates with the air bag 91, and a connection portion 93 provided at the leading end of the flow path body 92. One main surface of the air bag 91 of the sensing cuff 73 is fixed to the back plate 72. For example, the sensing cuff 73 is joined to the main surface of the back plate 72 on the wrist 200 side by the joining layer 75. The sensing cuff 73 with such a configuration is constituted by welding two sheet members 96.

Here, the air bags 91 are bag-like structures, and in the present embodiment, the blood pressure measurement device 1 is configured to use air with the pump, and thus the present embodiment will be described using the air bags. However, in a case where a fluid other than air is used, the bag-like structures may be fluid bags that are inflated by the fluid.

The air bag 91 is constituted in a rectangular shape that is long in one direction. The air bag 91 is constituted by, for example, combining two sheet members 96 long in one direction and, as illustrated in FIGS. 5 to 7, welding weld portions 91a using heat into a rectangular frame shape long in one direction.

The flow path body 92 is integrally provided at a portion of one edge portion of the air bag 91 in the longitudinal direction. As a specific example, the flow path body 92 is provided at the end portion of the air bag 91 near the device body 3. Additionally, the flow path body 92 is formed in a shape that is long in one direction and has less width than the width of the air bag 91 in the lateral direction, and formed with a leading end having a circular shape. The flow path body 92 includes the connection portion 93 on the leading end. The flow path body 92 is connected to a flow path portion 15 through the connection portion 93 and constitutes a flow path between the flow path portion 15 of the device body 3 and the air bag 91.

The flow path body 92 is constituted by welding a portion of sheet members 96, which is adjacent to a region of the sheet members 96 constituting the air bag 91, in a frame shape long in one direction using heat, in a state where the connection portion 93 is disposed on the two sheet members 96. A portion of the flow path body 92 is disposed, for example, from the inner circumferential surface side of the pressing cuff 71 to the curler 5 side through the insertion portion 71c of the pressing cuff 71. Note that, portions of the weld portions 91a where the two sheet members 96 are welded in a rectangular frame shape are not welded and the air bag 91 is constituted to be continuous with a weld portion 92a constituting the flow path body 92, and thus the air bag 91 fluidly communicates with the flow path body 92.

The connection portion 93 is, for example, a nipple. The connection portion 93 is provided at the leading end of the flow path body 92. Also, the leading end of the connection portion 93 is externally exposed from the sheet member 96 facing the curler 5 and the back plate 72, of the two sheet members 96 constituting the flow path body 92. The connection portion 93 is connected to the flow path portion.

As a specific example, the sensing cuff 73 includes a fifth sheet member 96a and a sixth sheet member 96b in this order from the wrist 200 side as illustrated in FIGS. 3 and 7. Note that the sensing cuff 73 is constituted by joining adjacent sheet members 96 by welding using heat.

For example, the fifth sheet member 96a and the sixth sheet member 96b are constituted in a shape that allows the air bag 91 and the flow path body 92 to be constituted. The air bag 91 and the flow path body 92 are constituted by the fifth sheet member 96a and the sixth sheet member 96b being disposed facing one another, welded using heat along the peripheral edge shapes of the air bag 91 and the flow path body 92 so that the air bag 91 fluidly communicates with the flow path body 92, and cut in a predetermined shape.

Furthermore, the sixth sheet member 96b includes a hole portion 96b1 into which the leading end of the connection portion 93 can be inserted, for example. In the sixth sheet member 96b, the connection portion 93 is disposed in the hole portion 96b1, and a periphery of the hole portion 96b1 is thermally welded to the connection portion 93. The sixth sheet member 96b is joined to the inner circumferential surface of the back plate 72 with the joining layer 75 in between.

Next, an example of the user attaching the blood pressure measurement device 1 to the wrist 200 will be described. FIGS. 8 to 10 illustrate an example of the user wearing the blood pressure measurement device 1 on the wrist 200.

As illustrated in FIG. 8, first, the user inserts the wrist 200 into the curler 5, for example. At this time, the portion protruding from the second end 5b of the curler 5 of the pressing cuff 71 possibly suspends downward depending on, for example, the posture of the wrist 200.

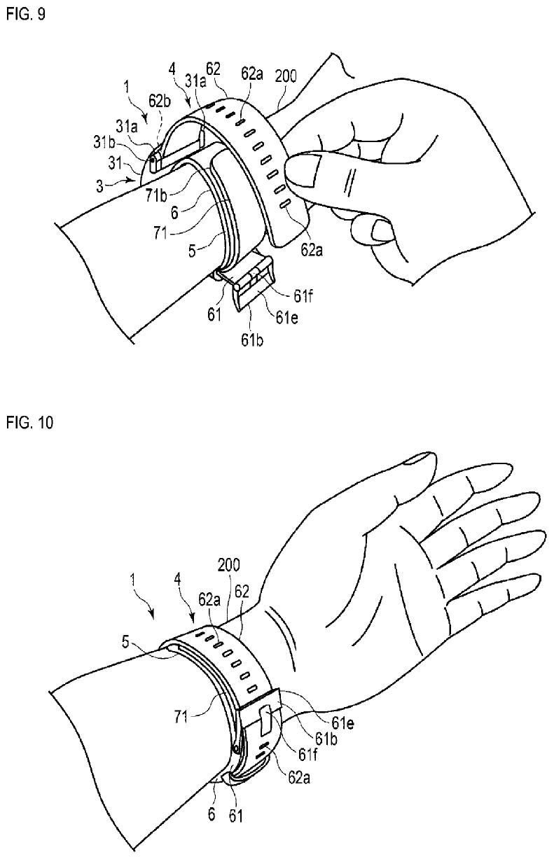

Next, as illustrated in FIG. 9, the user moves the portion protruding from the second end 5b of the curler 5 of the pressing cuff 71 to the outer circumferential surface 5d side of the curler 5. The user passes the second belt 62 through the frame body 61e of the buckle 61b of the first belt 61 with the hand different from the hand on which the blood pressure measurement device 1 is disposed. The user then pulls the second belt 62 to bring the member on the inner circumferential surface 5c side of the curler 5, namely, the cuff structure 6, into close contact with the wrist 200, and inserts the prong 61f into the small hole 62a.

At this time, the portion protruding from the second end 5b of the curler 5 of the pressing cuff 71 is disposed in the region where at least the sensing cuff 73 is present in the outer circumferential surface 5d of the curler 5, and is sandwiched by the curler 5 and the belt 4.

The blood pressure measurement device 1 is thus attached to the wrist 200 by this procedure.

In the blood pressure measurement device 1 configured in this manner, as illustrated in FIG. 2, in a state where the blood pressure measurement device 1 is attached to the wrist 200 having the assumed shortest circumference of the wrist 200, the sensing cuff 73 contacts the radial artery 211 and the ulnar artery 212 of the wrist 200. Furthermore, a portion of the portion protruding from the second end 5b of the curler 5 of the pressing cuff 71 is disposed in the region where the sensing cuff 73 is present in the outer circumferential surface 5d of the curler 5.

When the pressing cuff 71 is inflated in this state, the sensing cuff 73 is pressed to the wrist 200 side by the pressing cuff 71 disposed between the sensing cuff 73 and the curler 5. Furthermore, the sensing cuff 73 is pressed to the wrist 200 side by the pressing cuff 71 disposed in the region where the sensing cuff 73 is present in the outer circumferential surface 5d of the curler 5. Thus, a pressing force of pressing the sensing cuff 73 to the wrist 200 side can be increased, and thus the sensing cuff 73 is brought into close contact with the region where the radial artery 211 and the ulnar artery 212 are present in the wrist 200.

As illustrated in FIG. 12, in a state where the blood pressure measurement device 1 is attached to the wrist 200 having the assumed longest circumference of the wrist 200, the position of the sensing cuff 73 with respect to the wrist 200 is shifted to the radial artery 211 side with respect to the wrist 200 having the assumed shortest circumference. However, the sensing cuff 73 has the length at which the sensing cuff 73 contacts the region where the radial artery 211 is present with the blood pressure measurement device 1 attached to the wrist 200 having the assumed longest circumference of the wrist 200. Accordingly, the sensing cuff 73 is disposed in the region where the radial artery 211 is present in the wrist 200. Furthermore, the portion protruding from the second end 5b of the curler 5 of the pressing cuff 71 is disposed in the region where at least the sensing cuff 73 is present in the outer circumferential surface 5d of the curler 5.

When the pressing cuff 71 is inflated in this state, the sensing cuff 73 is pressed to the wrist 200 side by the pressing cuff 71 disposed between the curler 5 and the sensing cuff 73. Furthermore, the sensing cuff 73 is pressed to the wrist 200 side by the pressing cuff 71 disposed in the region where the sensing cuff 73 is present in the outer circumferential surface 5d of the curler 5. Thus, a pressing force of pressing the sensing cuff 73 against the wrist 200 can be increased, and thus the sensing cuff 73 is brought into close contact with the region where the artery 210 is present in the wrist 200.

Note that when the blood pressure measurement device 1 is attached to the wrist 200 having the relatively long circumference of the wrist 200, for example, when the blood pressure measurement device 1 is attached to the wrist 200 having the assumed longest circumference of the wrist 200, as the device body 3 indicated by the two-dot chain line in FIG. 12, the position in the circumferential direction of the wrist 200 of the blood pressure measurement device 1 may be adjusted. In this case, for example, the position in the circumferential direction of the wrist 200 of the blood pressure measurement device 1 may be adjusted so that the central portion in the circumferential direction of the sensing cuff 73 faces the radial artery 211.

Furthermore, the pressing cuff 71 has the length at which the pressing cuff 71 faces the region where at least the sensing cuff 73 is present in the outer circumferential surface 5d of the curler 5 when the blood pressure measurement device 1 is attached to the wrist 200 having any circumference from the assumed shortest circumference to the assumed longest circumference of the wrist 200.

As a result, the sensing cuff 73 can be brought into close contact with the region where the artery is present in the wrist 200 even with the wrist 200 having any circumference from the assumed shortest circumference to the longest circumference of the wrist 200.

Furthermore, the pressing cuff 71 is used as the cuff disposed between the sensing cuff 73 and the curler 5 and the cuff disposed in the region where the sensing cuff 73 is present in the curler 5. The use of one cuff in this manner allows preventing an increase in the number of components of the blood pressure measurement device 1.

As described above, the blood pressure measurement device 1 according to the present embodiment allows the sensing cuff to be brought into close contact with the region where the artery is present in the wrist 200.

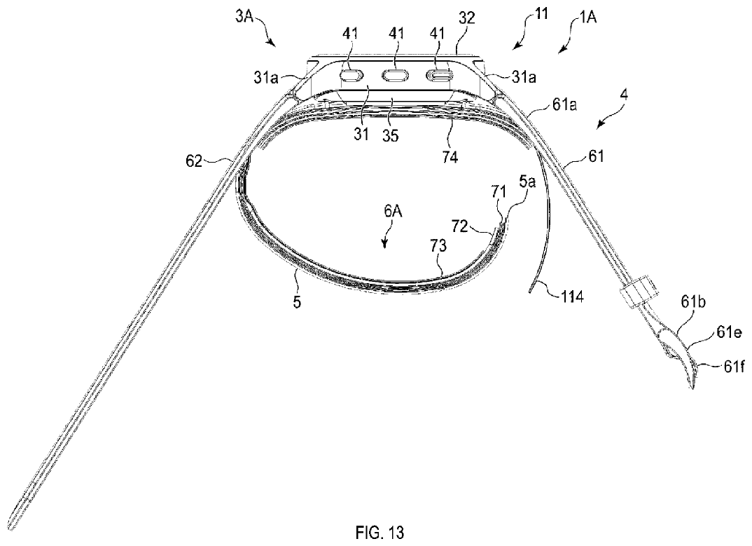

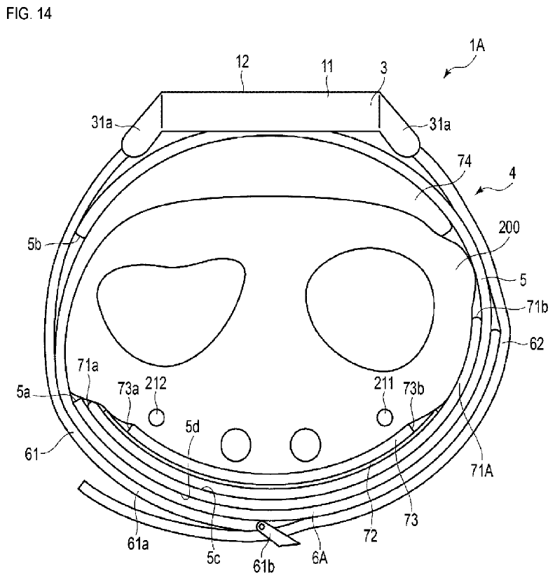

Next, an example of a blood pressure measurement device 1A according to a second embodiment of the present invention will be described below using FIGS. 13 to 18. Note that in the present embodiment, the configurations similar to those of the first embodiment are denoted by the same reference numerals as those of the first embodiment, and descriptions thereof are omitted.

FIG. 13 is a side view illustrating the configuration of the blood pressure measurement device 1A. FIG. 14 is an explanatory diagram illustrating a state where the blood pressure measurement device 1A is attached to the wrist 200. The wrist 200 illustrated in FIG. 14 is the wrist 200 of the user having the shortest circumference of the wrist 200 among the plurality of users set as targets using the blood pressure measurement device 1A. A cuff structure 6A illustrated in FIG. 14 is in an inflated state.

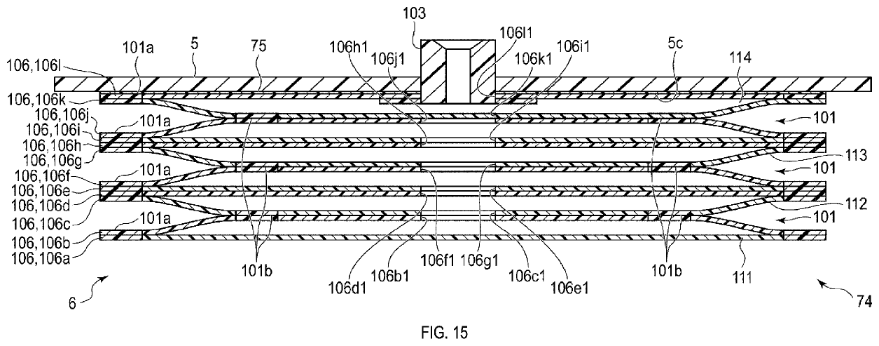

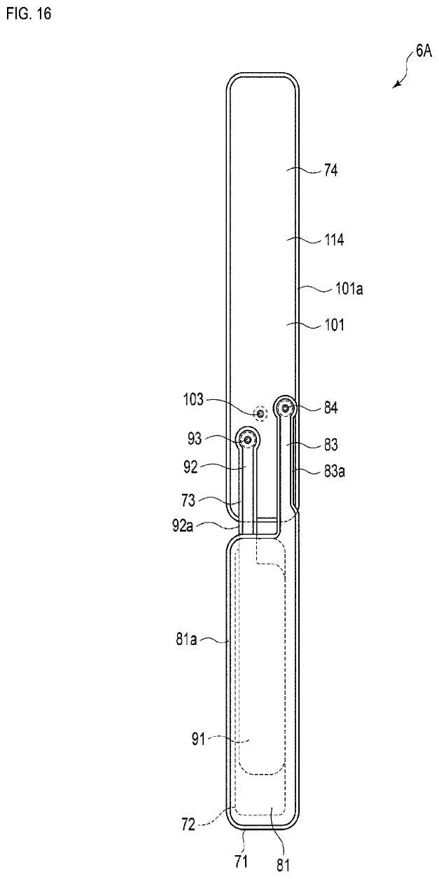

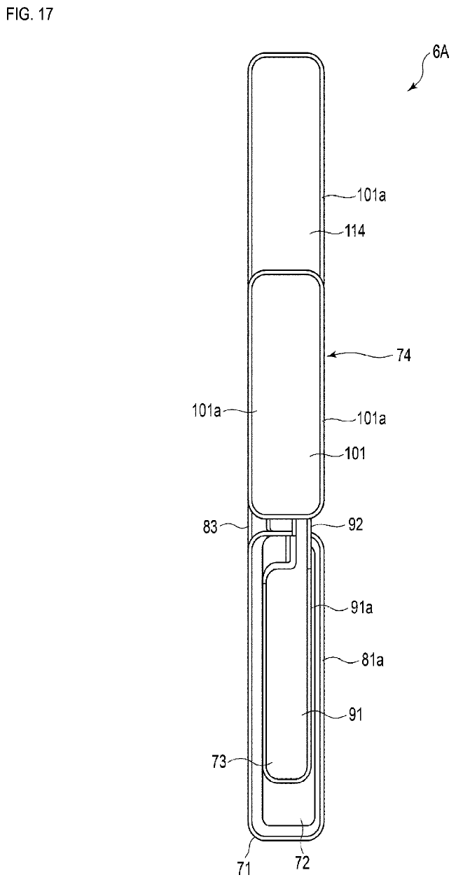

FIG. 15 is a cross-sectional view illustrating the configuration of the cuff structure 6A and the curler 5. FIG. 16 is a plan view illustrating a configuration of a surface on a side opposite to a surface fixed to the curler 5 of the cuff structure 6A. FIG. 17 is a plan view illustrating the configuration of the surface on the side fixed to the curler 5 of the cuff structure 6A.

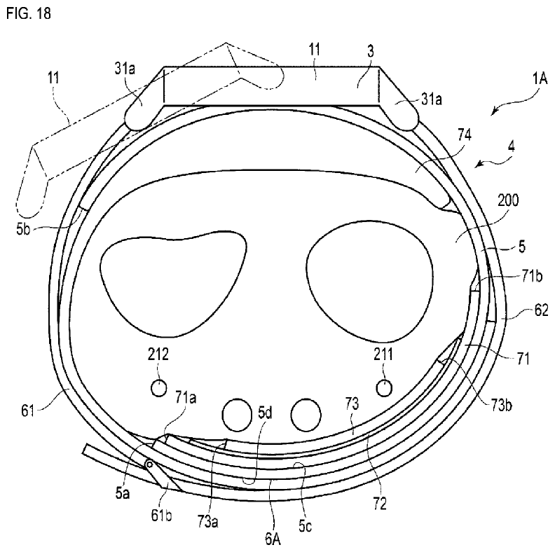

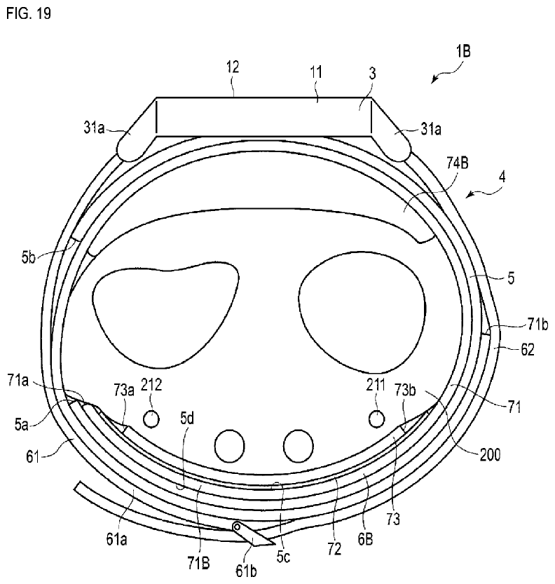

FIG. 18 is an explanatory diagram illustrating a state where the blood pressure measurement device 1A is attached to the wrist 200. The wrist 200 illustrated in FIG. 18 is the wrist 200 having the assumed longest circumference of the wrist 200 among the wrists 200 of the plurality of users set as targets using the blood pressure measurement device 1A. The cuff structure 6A illustrated in FIG. 18 is in an inflated state.

As illustrated in FIG. 13, the blood pressure measurement device 1A is an electronic blood pressure measurement device attached to a living body. In the present embodiment, the blood pressure measurement device 1A will be described using an electronic blood pressure measurement device having an aspect of a wearable device attached to the wrist 200 of the living body.

As illustrated in FIG. 13, the blood pressure measurement device 1A includes a device body 3A, the belt 4, the curler 5, and the cuff structure 6A.

In the curler 5, the cuff structure 6A is disposed on the inner circumferential surface. The curler 5 holds the cuff structure 6A along the shape of the inner circumferential surface 5c of the curler 5. For example, the cuff structure 6A is held by fixing the cuff structure 6A by the joining layer 75 provided between the curler 5 and the cuff structure 6A.

In the present embodiment, as illustrated in FIGS. 13 and 14, the cuff structure 6A includes a pressing cuff 71A (a first cuff), the back plate 72, the sensing cuff 73, and a tensile cuff (a second cuff) 74. The cuff structure 6A is provided with the joining layer 75 for joining respective mutual components and the curler 5 and the pressing cuff 71A together.

The pressing cuff 71A is fluidly connected to the pump via the flow path portion. The pressing cuff 71A inflates to press the back plate 72 and the sensing cuff 73 to the wrist 200 side. The pressing cuff 71A is configured in a band-like shape extending in one direction. The pressing cuff 71A is fixed to the inner circumferential surface of the curler 5 by the joining layer 75.

The first end 71a of the pressing cuff 71A is disposed in the vicinity of the first end 5a located on the hand palm side of the wrist 200 of the curler 5. The pressing cuff 71A has a configuration in which the pressing cuff 71 of the first embodiment does not include a portion disposed in the region where at least the sensing cuff 73 is present in the outer circumferential surface 5d of the curler 5. The pressing cuff 71A is not configured to protrude beyond the second end 5b of the curler 5.

The pressing cuff 71A specifically includes the plurality of air bags 81, the flow path body 83 that communicates with the air bag 81, and the connection portion 84 provided at the leading end of the flow path body 83. The plurality of air bags 81 are, for example, the two-layer air bags 81. The two-layer air bags 81 of the pressing cuff 71A have a size in which the air bag 81 faces the region of facing the hand palm side of the wrist 200 in the inner circumferential surface 5c of the curler 5.

As illustrated in FIG. 16, the flow path body 83 is integrally provided on the single air bag 81, for example, on a portion of the edge portion at the first end in the longitudinal direction of the air bag 81 adjacent to the curler 5. As a specific example, the flow path body 83 is provided at the end portion of the air bag 81 near the device body 3. Additionally, the flow path body 83 is formed in a shape that is long in one direction and has less width than the width of the air bag 81 in the lateral direction and formed with a leading end having a circular shape. The flow path body 83 includes the connection portion 84 on the leading end.

The flow path body 83 is constituted by welding a portion of sheet members 86, which is adjacent to a region of the sheet members 86 constituting the air bags 81, in a frame shape long in one direction using heat, in a state where the connection portion 84 is disposed on the two sheet members 86.

Note that, portions of the weld portions 81a where the two sheet members 86 are welded in a rectangular frame shape are not welded and the air bag 81 in which the flow path body 83 is provided is constituted to be continuous with a weld portion 83a constituting the flow path body 83, and thus the air bag 81 fluidly communicates with the flow path body 83. The connection portion 84 is connected to the flow path portion.

As illustrated in FIGS. 13 to 15, the tensile cuff 74 is fluidly connected to the pump via the flow path portion. The tensile cuff 74 is fixed to the hand back side of the wrist 200 of the curler 5. In addition, at least a portion of the tensile cuff 74 protrudes from the second end 5b of the curler 5 and is disposed in a region where the sensing cuff 73 is present in the outer circumferential surface 5d of the curler 5 with the blood pressure measurement device 1A attached.

The tensile cuff 74 is inflated to press the curler 5 such that the curler 5 is spaced apart from the wrist 200, pulling the belt 4 and the curler 5 toward the hand back side of the wrist 200. The tensile cuff 74 includes, for example, a plurality of air bags 101 and a connection portion 103 provided in the air bag 101 facing the curler 5. The plurality of air bags 101 are, for example, the six-layer air bags 101.

The tensile cuff 74 with such a configuration is constituted by welding a plurality of sheet members 106. In addition, the tensile cuff 74 is fixed to the hand back side of the wrist 200 of the curler 5. In other words, the flow path body 83 of the pressing cuff 71 and the flow path body 92 of the sensing cuff 73 are disposed between the hand back side of the wrist 200 of the curler 5 and the tensile cuff 74.

Additionally, the tensile cuff 74 is configured such that the thickness of the tensile cuff 74 in an inflating direction, in the present embodiment, in the direction in which the curler 5 and the wrist 200 face one another, during inflation, is larger than the thickness of the pressing cuff 71A in the inflating direction during inflation and than the thickness of the sensing cuff 73 in the inflating direction during inflation. Specifically, the air bags 101 of the tensile cuff 74 include more layer structures than the air bags 81 in the pressing cuff 71A and the air bag 91 in the sensing cuff 73, and have thicker thickness than the pressing cuff 71A and the sensing cuff 73 when the air bags 101 are inflated from the curler 5 toward the wrist 200.

In the present embodiment, the tensile cuff 74 including the six-layer air bags 101 includes a first outer layer 111 constituted by one air bag 101, a first intermediate layer 112 constituted by two air bags 101 integrally combining with the first outer layer 111 by welding using heat, a second intermediate layer 113 constituted by two-layer air bags 101 integrally combining with the first intermediate layer 112 by welding using heat, and a second outer layer 114 constituted by one air bag 101 integrally combining with the second intermediate layer 113 by welding using heat.

Here, the air bags 101 are bag-like structures, and in the present embodiment, the blood pressure measurement device 1A is configured to use air with the pump, and thus the present embodiment will be described using the air bags. However, in a case where a fluid other than air is used, the bag-like structures may be fluid bags that are inflated by the fluid. A plurality of the air bags 101 are stacked and are in fluid communication in the stacking direction.

Each of the air bags 101 is formed in a rectangular bag-like shape that is long in one direction. Additionally, the air bags 101 are set so that the width in the lateral direction is the same as the width in the lateral direction of the curler 5. The air bag 101 is constituted by, for example, combining two sheet members 106 and, as illustrated in FIGS. 16 and 17, welding weld portions 101a using heat into a rectangular frame shape long in one direction. The six-layer air bags 101 fluidly communicate with openings provided in the sheet members 106 facing one another.

In addition, in the six-layer air bags 101, for the first outer layer 111 and the first intermediate layer 112, the first intermediate layer 112 and the second intermediate layer 113, and the second intermediate layer 113 and the second outer layer 114, by bridge welding the respective facing sheet members 106 together in a quadrilateral frame shape smaller than the weld portions 81a located on the outer peripheral edges and surrounding the plurality of openings with bridge weld portions 101b, the adjacent air bags 101 are integrally formed and fluidly communicate at the inner sides of the bridge weld portions 101b.

The first outer layer 111 is formed by one air bag 101 disposed on the wrist 200 side. The first outer layer 111 constitutes the first air bag 101 of the six-layer air bags 101 from the wrist 200 side.

The first intermediate layer 112 is stacked on the first outer layer 111. The first intermediate layer 112 is formed by two-layer air bags 101. The first intermediate layer 112 constitutes the second and third air bags 101 of the six-layer air bags 101 from the wrist 200 side. The first intermediate layer 112 is constituted by two-layer air bags 101 integrally welded at the outer peripheral edge. In other words, the first intermediate layer 112 is formed by integrally welding four sheet members 106 in the outer peripheral edge shape of the air bags 101.

The second intermediate layer 113 is stacked on the first intermediate layer 112. The second intermediate layer 113 is formed by two-layer air bags 101. The second intermediate layer 113 constitutes the fourth and fifth air bags 101 of the six-layer air bags 101 from the wrist 200 side. The second intermediate layer 113 is constituted by two-layer air bags 101 integrally welded at the outer peripheral edge. In other words, the second intermediate layer 113 is formed by integrally welding four sheet members 106 in the outer peripheral edge shape of the air bags 101.

The second outer layer 114 is formed by one air bag 101 disposed on the curler 5 side. The second outer layer 114 constitutes the sixth air bag 101 of the six-layer air bags 101 from the wrist 200 side.

The connection portion 103 is, for example, a nipple. The connection portion 103 is provided on the air bag 101 disposed adjacent to the curler 5. The leading end of the connection portion 103 is exposed from the sheet member 106 facing the curler 5, of the two sheet members 106 forming the air bag 101. The connection portion 103 is connected to the flow path portion.

As a specific example, as illustrated in FIG. 15, the tensile cuff 74 includes a seventh sheet member 106a, an eighth sheet member 106b, a ninth sheet member 106c, a tenth sheet member 106d, an eleventh sheet member 106e, a twelfth sheet member 106f, a thirteenth sheet member 106g, a fourteenth sheet member 106h, a fifteenth sheet member 106i, a sixteenth sheet member 106j, a seventeenth sheet member 106k, and an eighteenth sheet member 106l in this order from the wrist 200 side. Note that the tensile cuff 74 is integrally constituted by joining adjacent sheet members 106 by welding using heat.

The seventh sheet member 106a to the eighteenth sheet member 106l are constituted in a similar rectangular shape to the air bags 101. The seventh sheet member 106a and the eighth sheet member 106b are welded using heat along the peripheral edge portion shape on the four sides of the air bag 101 to constitute the air bag 101 of the first layer from the wrist 200 side. In other words, the seventh sheet member 106a and the eighth sheet member 106b constitute the first outer layer 111.

The eighth sheet member 106b and the ninth sheet member 106c are disposed facing one another, and include a plurality of openings 106b1 and 106c1, respectively, through which the two air bags 101 fluidly communicate with one another. Additionally, the eighth sheet member 106b and the ninth sheet member 106c are integrally joined by the peripheral region of the plurality of openings 106b1 and 106c1 being bridge welded using heat in a quadrilateral frame shape smaller than the welded four sides of the air bags 101.

The ninth sheet member 106c and the tenth sheet member 106d are welded using heat along the peripheral edge portion shape on the four sides of the air bag 101 to constitute the air bag 101 of the second layer from the wrist 200 side.

The tenth sheet member 106d and the eleventh sheet member 106e are disposed facing one another, and include a plurality of openings 106d1 and 106e1, respectively, through which the two air bags 101 fluidly communicate with one another. The eleventh sheet member 106e and the twelfth sheet member 106f are welded using heat along the peripheral edge portion shape on the four sides of the air bag 101 to constitute the air bag 101 of the third layer from the wrist 200 side.

The ninth sheet member 106c, the tenth sheet member 106d, the eleventh sheet member 106e, and the twelfth sheet member 106f are integrally welded using heat along the peripheral edge portion shape on the four sides of the air bags 101 to constitute the first intermediate layer 112 in which the second and third air bags 101 are integrally formed.

The twelfth sheet member 106f and the thirteenth sheet member 106g are disposed facing one another, and include a plurality of openings 106f1 and 106g1, respectively, through which the two air bags 101 fluidly communicate with one another. Additionally, the twelfth sheet member 106f and the thirteenth sheet member 106g are integrally joined by the peripheral region of the plurality of openings 106f1 and 106g1 being bridge welded using heat in a quadrilateral frame shape smaller than the welded four sides of the air bags 101.

The thirteenth sheet member 106g and the fourteenth sheet member 106h are welded using heat along the peripheral edge portion shape on the four sides of the air bag 101 to constitute the air bag 101 of the fourth layer from the wrist 200 side.

The fourteenth sheet member 106h and the fifteenth sheet member 106i are disposed facing one another, and include a plurality of openings 106h1 and 106i1, respectively, through which the two air bags 101 fluidly communicate with one another. The fifteenth sheet member 106i and the sixteenth sheet member 106j are welded using heat along the peripheral edge portion shape on the four sides of the air bag 101 to constitute the air bag 101 of the fifth layer from the wrist 200 side.

The thirteenth sheet member 106g, the fourteenth sheet member 106h, the fifteenth sheet member 106i, and the sixteenth sheet member 106j are integrally welded using heat along the peripheral edge portion shape on the four sides of the air bags 101 to constitute the second intermediate layer 113 in which the fourth and fifth air bags 101 are integrally formed.

The sixteenth sheet member 106j and the seventeenth sheet member 106k are disposed facing one another, and include a plurality of openings 106j1 and 106k1, respectively, through which the two air bags 101 fluidly communicate with one another. Further, the seventeenth sheet member 106k is constituted in a shape capable of constituting the air bag 101, for example. Additionally, the sixteenth sheet member 106j and the seventeenth sheet member 106k are integrally joined by the peripheral region of the plurality of openings 106j1 and 106k1 being bridge welded using heat in a quadrilateral frame shape smaller than the welded four sides of the air bags 101.

The seventeenth sheet member 106k and the eighteenth sheet member 106l are thermally welded along the peripheral edge portion shape of the four sides of the air bag 101 and cut into a predetermined shape to constitute the air bag 101.

Furthermore, the eighteenth sheet member 106l includes a hole portion 106l1 into which the leading end of the connection portion 103 can be inserted, for example. The eighteenth sheet member 106l is disposed with the connection portion 103 at the hole portion 106l1, and the peripheral region of the hole portion 106l1 is welded to the connection portion 103 using heat. Further, the eighteenth sheet member 106l is joined to the inner circumferential surface 5c of the curler 5 via the joining layer 75, and the seventeenth sheet member 106k is joined to the inner circumferential surface 5c of the curler 5 via the joining layer 75.

At least a portion of the tensile cuff 74 configured in this manner protrudes from the second end 5b of the curler 5 along the circumferential direction of the curler 5. The protruding portion is configured to have a length such that a portion of the protruding portion is disposed in the region where the sensing cuff 73 is present in the outer circumferential surface 5d of the curler 5 when the blood pressure measurement device 1A is attached to the wrist 200 having any circumference from the assumed shortest circumference to the assumed longest circumference of the wrist 200.

For example, as illustrated in FIGS. 16 and 17, the second outer layer 114 of the tensile cuff 74 is formed longer than the other layers of the tensile cuff 74, and the second outer layer 114 is disposed in the region where at least the sensing cuff 73 is present in the outer circumferential surface 5d of the curler 5 when the blood pressure measurement device 1A is attached to the wrist 200 having any circumference from the assumed shortest circumference to the assumed longest circumference of the wrist 200.

Specifically, as illustrated in FIG. 14, in a state where the device body 3A is disposed on the hand back side of the wrist 200, the second outer layer 114 has the length extending to the lateral side of the wrist 200 beyond the sensing cuff 73 and the radial artery 211 with respect to the wrist 200 having the assumed shortest circumference of the wrist 200. Further, as illustrated in FIG. 18, the second outer layer 114 has the length extending to the lateral side of the wrist 200 beyond the sensing cuff 73 and the radial artery 211 with respect to the wrist 200 having the assumed longest circumference of the wrist 200.

As illustrated in FIG. 14, in the blood pressure measurement device 1A configured in this manner, in a state where the blood pressure measurement device 1A is attached to the wrist 200 having the assumed shortest circumference of the wrist 200, the sensing cuff 73 contacts the region where the radial artery 211 and the ulnar artery 212 are present in the wrist 200, and a portion of the second outer layer 114 of the tensile cuff 74 is disposed in the region where the sensing cuff 73 is present in the outer circumferential surface 5d of the curler 5. When the pressing cuff 71 is inflated in this state, the sensing cuff 73 is pressed to the wrist 200 side by the pressing cuff 71. When the tensile cuff 74 is inflated, the outer circumferential surface 5d of the curler 5 is pressed by the second outer layer 114 of the tensile cuff 74, and thus the sensing cuff 73 is pressed to the wrist 200 side. In this manner, the sensing cuff 73 is pressed by the pressing cuff 71 and the second outer layer 114 of the tensile cuff 74, thereby ensuring increasing the pressing force that presses the sensing cuff 73 against the wrist 200, and thus the sensing cuff 73 is brought into close contact with the region where the radial artery 211 and the ulnar artery 212 are present in the wrist 200.

As illustrated in FIG. 18, in a state where the blood pressure measurement device 1A is attached to the wrist 200 having the assumed longest circumference of the wrist 200, the sensing cuff 73 contacts the region where the radial artery 211 is present in the wrist 200, and a portion of the second outer layer 114 of the tensile cuff 74 is disposed in the region where the sensing cuff 73 is present in the outer circumferential surface 5d of the curler 5.

When the pressing cuff 71 is inflated in this state, the sensing cuff 73 is pressed to the wrist 200 side by the pressing cuff 71. When the tensile cuff 74 is inflated, the outer circumferential surface 5d of the curler 5 is pressed by the second outer layer 114 of the tensile cuff 74, and thus the sensing cuff 73 is pressed to the wrist 200 side. In this manner, the sensing cuff 73 is pressed by the pressing cuff 71 and the second outer layer 114 of the tensile cuff 74, thereby ensuring increasing the pressing force that presses the sensing cuff 73 against the wrist 200, and thus the sensing cuff 73 is brought into close contact with the region where the radial artery 211 is present in the wrist 200.

Note that when the blood pressure measurement device 1A is attached to the wrist 200 having the relatively long circumference of the wrist 200, for example, when the blood pressure measurement device 1A is attached to the wrist 200 having the assumed longest circumference of the wrist 200, as the device body 3 indicated by the two-dot chain line in FIG. 18, the position in the circumferential direction of the wrist 200 of the blood pressure measurement device 1A may be adjusted. In this case, for example, the position in the circumferential direction of the wrist 200 of the blood pressure measurement device 1A may be adjusted so that the central portion in the circumferential direction of the sensing cuff 73 faces the radial artery 211.

The blood pressure measurement device 1A configured in this manner includes the pressing cuff 71A as the cuff disposed between the sensing cuff 73 and the curler 5 and includes the tensile cuff 74 as the cuff disposed in the region where at least the sensing cuff 73 is present in the outer circumferential surface 5d of the curler 5 with the blood pressure measurement device 1A attached to the wrist 200. Thus, the sensing cuff 73 can be in close contact with the region where the artery 210 is present in the wrist 200.

Furthermore, the second outer layer 114 of the tensile cuff 74 has the length at which the second outer layer 114 faces the region where at least the sensing cuff 73 is present in the outer circumferential surface 5d of the curler 5 when the blood pressure measurement device 1A is attached to the wrist 200 having any circumference from the assumed shortest circumference to the assumed longest circumference of the wrist 200.

Thus, the sensing cuff 73 can be brought into close contact with the region where the artery is present in the wrist 200 in the wrist 200 having any circumference from the assumed shortest circumference to the assumed longest circumference of the wrist 200.

Furthermore, in the configuration provided with the tensile cuff 74, the use of the tensile cuff 74 as the cuff disposed in the region where at least the sensing cuff 73 is present in the outer circumferential surface 5d of the curler 5 allows preventing the increase in the number of components of the blood pressure measurement device 1A.

As described above, the blood pressure measurement device 1A according to the present embodiment allows the sensing cuff to be brought into close contact with the region where the artery is present in the wrist 200.

A blood pressure measurement device 1B according to a third embodiment of the present invention will be described below using FIGS. 19 to 21. Note that the configurations similar to those of the first embodiment and the configurations similar to those of the second embodiment are denoted by the same reference numerals as those of the first embodiment and the second embodiment, and descriptions thereof are omitted.

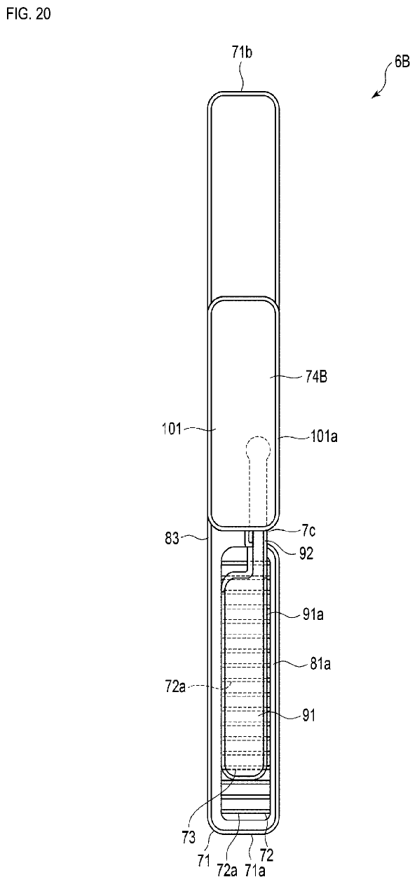

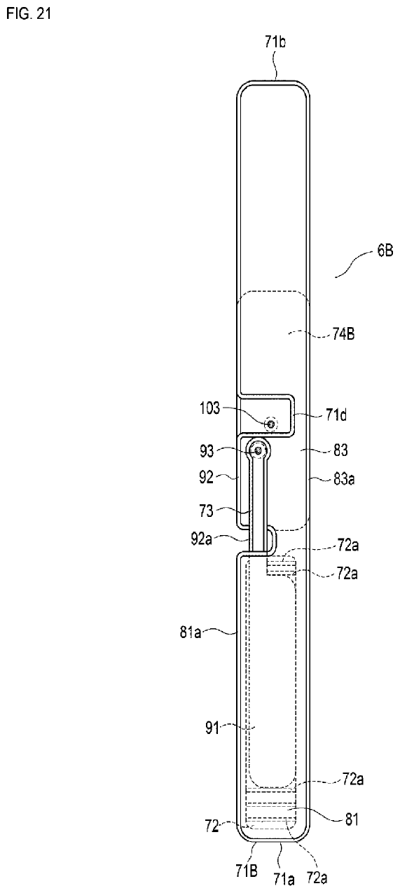

FIG. 19 is an explanatory diagram illustrating a state where the blood pressure measurement device 1B is attached to the wrist 200. The wrist 200 illustrated in FIG. 19 is the wrist 200 having the assumed shortest circumference of the wrist 200 among the wrists 200 of the plurality of users set as targets using the blood pressure measurement device 1B. A cuff structure 6B illustrated in FIG. 19 is in an inflated state. FIG. 20 is a plan view illustrating a state of viewing the configuration of the cuff structure 6B from the wrist 200 side. FIG. 21 is a plan view illustrating a state of viewing the configuration of the cuff structure 6B from the inner circumferential surface 5c side of the curler 5.

As illustrated in FIG. 19, the blood pressure measurement device 1B includes the device body 3, the belt 4, the curler 5, and the cuff structure 6B provided on the curler 5.

In the curler 5, the cuff structure 6B is disposed on the inner circumferential surface. The curler 5 holds the cuff structure 6B along the shape of the inner circumferential surface 5c of the curler 5. For example, the cuff structure 6B is held by fixing the cuff structure 6B by the joining layer 75 provided between the curler 5 and the cuff structure 6B. In the present embodiment, the joining layer 75 is adhesive or double-sided tape.

The cuff structure 6B includes a pressing cuff 71B, the back plate 72, the sensing cuff 73, and a tensile cuff 74B.

The pressing cuff 71B differs from the pressing cuff 71 of the first embodiment in that the pressing cuff 71B includes an insertion portion 71d for connecting the connection portion 103 of the tensile cuff 74 to the flow path portion. The insertion portion 71d is configured in a region facing the connection portion 103 in the pressing cuff 71B. The other configurations of the pressing cuff 71B are the same as those of the pressing cuff 71. The insertion portion 71d is configured, for example, in a shape in which a portion of an edge along the longitudinal direction of the pressing cuff 71B is recessed toward the edge portion side along the second longitudinal direction in the lateral direction.

The tensile cuff 74B differs in that the second outer layer 114 is formed to have a length same as those of the other layers while a portion of the second outer layer 114 of the tensile cuff 74 of the second embodiment is disposed in the region where the sensing cuff 73 is present in the outer circumferential surface 5d of the curler 5. The other configurations of the tensile cuff 74B are the same as those of the tensile cuff 74 of the second embodiment.

Specifically, the length of the second outer layer 114 of the tensile cuff 74B along the circumferential direction of the curler 5 is the same as the length of the first outer layer 111 along the circumferential direction of the curler 5.

Note that the pressing cuff 71B of the present embodiment includes the insertion portion 71d for connecting the connection portion 103 of the tensile cuff 74 to the flow path portion. The insertion portion 71d is configured in a region facing the connection portion 103 in the pressing cuff 71B. The insertion portion 71d is configured, for example, in a shape in which a portion of an edge along the longitudinal direction of the pressing cuff 71B is recessed toward the edge portion side along the other longitudinal direction in the lateral direction.

In this way, with the cuff structure 6B provided with the pressing cuff 71B, inflation of the pressing cuff 71B allows the sensing cuff 73 to be pressed to the wrist 200 side by the pressing cuff 71B disposed between the curler 5 and the sensing cuff 73. Further, the outer circumferential surface 5d of the curler 5 is pressed by the pressing cuff 71B and thus the sensing cuff 73 can be pressed to the wrist 200 side. Thus, the same effects as those of the first embodiment are obtained.

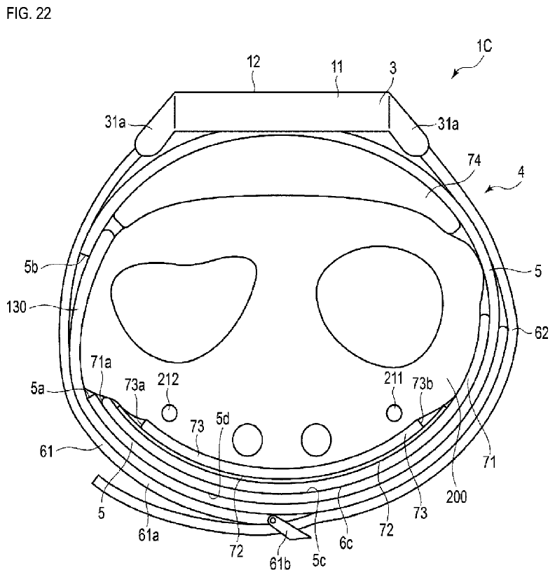

Next, a blood pressure measurement device 1C according to a fourth embodiment will be described using FIGS. 22 to 24. Note that configurations having the same functions as those of the second embodiment and configurations having the same functions as those of the third embodiment are denoted by the same reference numerals as those of the second embodiment and the third embodiment, and descriptions thereof are omitted.

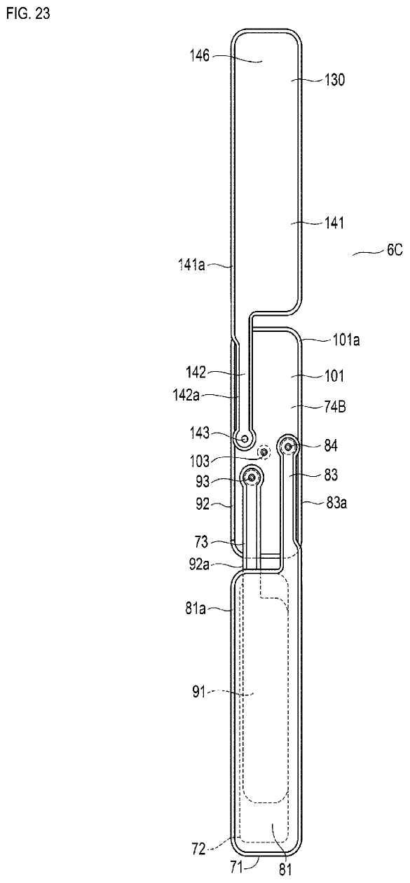

FIG. 22 is an explanatory diagram illustrating a state where the blood pressure measurement device 1C is attached to the wrist 200. FIG. 22 illustrates an example of a state where the blood pressure measurement device 1C is attached to the wrist 200 having the assumed shortest circumference among the wrists 200 of the plurality of users assumed as the targets using the blood pressure measurement device 1C. A cuff structure 6C illustrated in FIG. 22 is in an inflated state. FIG. 23 is a plan view illustrating a state of viewing the cuff structure 6C from the inner circumferential surface 5c side of the curler 5.

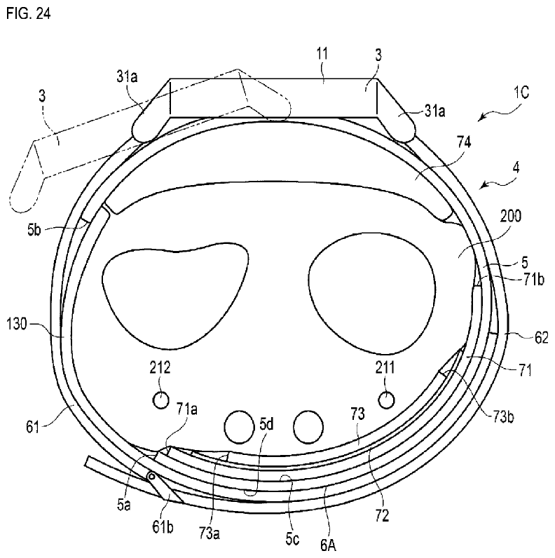

FIG. 24 is an explanatory diagram illustrating a state where the blood pressure measurement device 1C is attached to the wrist 200. FIG. 24 illustrates an example of a state where the blood pressure measurement device 1C is attached to the wrist 200 having the assumed longest circumference among the wrists 200 of the plurality of users assumed as the targets using the blood pressure measurement device 1C. The cuff structure 6C illustrated in FIG. 24 is in an inflated state.

As illustrated in FIG. 22, the blood pressure measurement device 1C includes the device body 3, the belt 4, the curler 5, and the cuff structure 6C provided on the curler 5.

In the curler 5, the cuff structure 6C is disposed on the inner circumferential surface. The curler 5 holds the cuff structure 6C along the shape of the inner circumferential surface 5c of the curler 5. For example, the curler 5 holds the cuff structure 6C by fixing the cuff structure 6C by the joining layer 75 provided between the curler 5 and the cuff structure 6C.

The cuff structure 6C includes the pressing cuff 71A (the first cuff), the back plate 72, the sensing cuff 73, the tensile cuff 74B, and a cuff 130 (a second cuff). A portion of the cuff 130 is disposed in a region where the sensing cuff 73 is present in the outer circumferential surface 5d of the curler 5.

As illustrated in FIGS. 22 and 23, the cuff 130 is disposed on a side opposite to the sensing cuff 73 with respect to the tensile cuff 74B in the circumferential direction of the curler 5. A portion of the cuff 130 is fixed to the inner circumferential surface 5c of the curler 5 by, for example, the joining layer 75.

As illustrated in FIG. 22, the cuff 130 is constituted in a band-like shape that extends along the circumferential direction of the curler 5. The cuff 130 protrudes from the second end 5b of the curler 5 in the circumferential direction of the curler 5. The length of the cuff 130 is set to be a length such that a portion of the cuff 130 is disposed in the region where the sensing cuff 73 is present in the outer circumferential surface 5d of the curler 5 when the blood pressure measurement device 1C is attached to the wrist 200 having any circumference from the assumed shortest circumference to the assumed longest circumference of the wrist 200.

As illustrated in FIG. 23, the cuff 130 includes, for example, one air bag 141, a flow path body 142 that communicates with the air bag 141, and a connection portion 143 provided at the leading end of the flow path body 142. The cuff 130 has one main surface of the air bag 141 joined to the inner circumferential surface 5c of the curler 5 by, for example, the joining layer 75. The cuff 130 with such a configuration is constituted by welding two sheet members 146.

Here, the air bags 141 are bag-like structures, and in the present embodiment, the blood pressure measurement device 1C is configured to use air with the pump, and thus the present embodiment will be described using the air bags. However, in a case where a fluid other than air is used, the bag-like structures may be fluid bags that are inflated by the fluid.

The air bag 141 is constituted in a rectangular shape that is long in one direction. The air bag 141 is constituted by, for example, combining the two sheet members 146 long in one direction and, as illustrated in FIG. 23, welding weld portions 141a using heat into a rectangular frame shape long in one direction.

The flow path body 142 is integrally provided at a portion of one edge portion of the air bag 141 in the longitudinal direction. As a specific example, the flow path body 142 is provided at the end portion of the air bag 141 near the device body 3. Additionally, the flow path body 142 is formed in a shape that is long in one direction and has a width smaller than the width of the air bag 91 in the lateral direction, and formed with a leading end having a circular shape. The flow path body 142 includes the connection portion 143 on the leading end. The flow path body 142 is connected to the flow path portion via the connection portion 143 and constitutes a flow path between the flow path portion 15 and the air bag 141.

The flow path body 142 is constituted by welding a portion of the sheet members 146 adjacent to regions of the sheet members 146 constituting the air bag 141 in a frame shape long in one direction using heat, in a state where the connection portion 143 is disposed on the two sheet members 146. Note that, portions of the weld portions 141a where the two sheet members 146 are welded in a rectangular frame shape are not welded and the air bag 141 is constituted to be continuous with a weld portion 142a constituting the flow path body 142, and thus the air bag 141 fluidly communicates with the flow path body 142.

The connection portion 143 is, for example, a nipple. The connection portion 143 is provided at the leading end of the flow path body 142. The connection portion 143 is connected to the flow path portion.

As illustrated in FIGS. 22 and 24, in the blood pressure measurement device 1C configured in this manner, a portion of the cuff 130 is disposed in the region where at least the sensing cuff 73 is present in the outer circumferential surface 5d of the curler 5 when the blood pressure measurement device 1C is attached to the wrist 200 having any circumference from the assumed shortest circumference to the assumed longest circumference of the wrist 200. The pressing cuff 71A is provided between the sensing cuff 73 and the curler 5.

The pressing cuff 71A disposed between the sensing cuff 73 and the curler 5 and the cuff 130 disposed on the outer circumferential surface 5d of the curler 5 are inflated to press the sensing cuff 73 against the wrist 200, thus allowing the sensing cuff 73 to be in close contact with the region where the artery is present in the wrist 200.

Thus, by pressing the sensing cuff 73 from the inner circumferential surface 5c side and the outer circumferential surface side of the curler 5, the pressing force that presses the sensing cuff 73 against the wrist 200 can be increased, and thus the sensing cuff 73 can be in close contact with the wrist 200.

Note that when the blood pressure measurement device 1C is attached to the wrist 200 having the relatively long circumference of the wrist 200, for example, when the blood pressure measurement device 1C is attached to the wrist 200 having the assumed longest circumference of the wrist 200, as the device body 3 indicated by the two-dot chain line in FIG. 24, the position of the blood pressure measurement device 1C in the circumferential direction of the wrist 200 may be adjusted. In this case, for example, the position in the circumferential direction of the wrist 200 of the blood pressure measurement device 1C may be adjusted so that the central portion in the circumferential direction of the sensing cuff 73 faces the radial artery 211

Furthermore, the sensing cuff 73 has the length along the curler 5 contacting the region where the artery is present in the wrist 200 when the blood pressure measurement device 1C is attached to the wrist 200 having any circumference from the assumed shortest circumference to the assumed longest circumference of the wrist 200. The cuff 130 has the length facing the region where the sensing cuff 73 is present in the outer circumferential surface 5d of the curler 5 in the wrist 200 having any circumference from the assumed shortest circumference to the assumed longest circumference.

This allows the sensing cuff 73 to be brought into close contact with the region where the artery is present in the wrist 200 in the wrist 200 having any length from the shortest to the longest circumference of the wrist 200.

As described above, the blood pressure measurement device 1C according to the present embodiment allows the sensing cuff to be brought into close contact with the region where the artery is present in the wrist 200.

Note that in the example described above, the configuration of the blood pressure measurement device 1, 1A, 1B, or 1C in which the curler 5 has the first end 5a and the second end 5b disposed on one lateral side of the wrist 200, the second end 5b is disposed on the hand back side, and the first end 5a is disposed on the hand palm side has been described as an example, but the configuration is not limited to this.

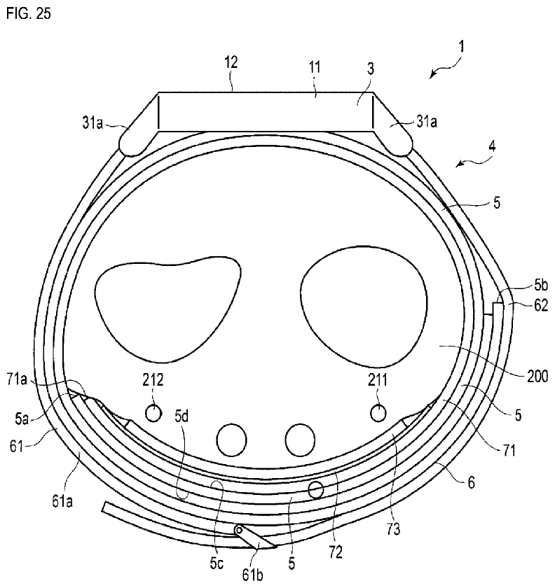

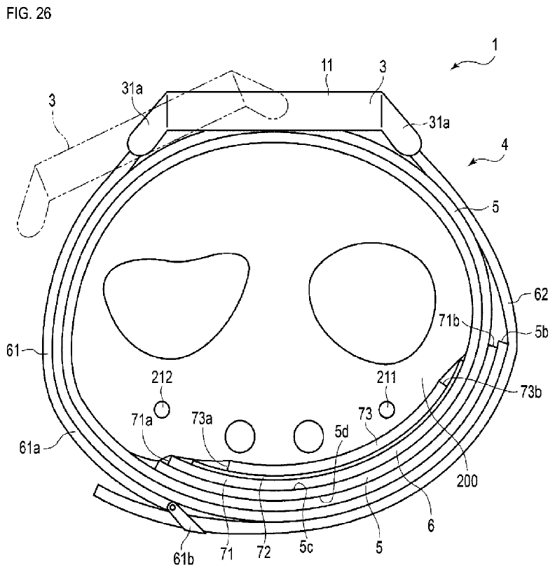

In another example, the curler 5 may have a length for which the cuff disposed in the region where the sensing cuff 73 is present in the outer circumferential surface 5d of the curler 5 on the inner circumferential surface. An example of a configuration that applies this modified example to the blood pressure measurement device 1 of the first embodiment will be described using FIGS. 25 and 26.

FIG. 25 is an explanatory diagram illustrating a state where the blood pressure measurement device 1 is attached to the wrist 200. The wrist 200 illustrated in FIG. 25 is the wrist 200 having the assumed shortest circumference as the circumference of the wrist 200 among the wrists 200 of the plurality of users set as targets using the blood pressure measurement device 1. The cuff structure 6 illustrated in FIG. 25 is in an inflated state.

FIG. 26 is an explanatory diagram illustrating a state where the blood pressure measurement device 1 is attached to the wrist 200. The wrist 200 illustrated in FIG. 26 is the wrist 200 having the assumed longest circumference of the wrist 200 among the wrists 200 of the plurality of users assumed as targets using the blood pressure measurement device 1. The cuff structure 6 illustrated in FIG. 26 is in an inflated state.

As illustrated in FIGS. 25 and 26, the curler 5 has a length such that a cuff disposed in a region where the sensing cuff 73 is present in the outer circumferential surface 5d of the curler 5 on the inner circumferential surface with the blood pressure measurement device 1 attached. In other words, in the curler 5, the first end 5a and the second end 5b are spaced apart, and the first end 5a overlaps with a portion of another portion of the curler 5.

The curler 5 has, for example, a length at which the end portion on the second end 5b side is located at a position in a region where the sensing cuff 73 is present in the outer circumferential surface 5d of the curler 5 or beyond the region in the circumferential direction of the curler 5, and the end portion overlaps with a portion of the other portion of the curler 5 in a direction from the curler 5 toward the wrist 200. Furthermore, the cuff disposed on the outer circumferential surface 5d of the curler 5 has a length so as not to project from the curler 5.

According to the curler 5 configured in this manner, in a state where the blood pressure measurement device 1 is attached to the wrist 200, the cuff 71 disposed in the region where the sensing cuff 73 is present in the outer circumferential surface 5d of the curler 5 is supported by the inner circumferential surface 5c of the curler 5 disposed further outward of the cuff 71. Thus, the direction of inflation of the cuff disposed in the region where the sensing cuff 73 is present in the outer circumferential surface 5d of the curler 5 is a direction toward the wrist 200 side. That is, the curler 5 can suppress that the inflation direction of the pressing cuff 71 from being in the direction in which the pressing cuff 71 becomes out of the inner circumferential surface of the curler 5. Accordingly, inflation of the pressing cuff 71 can be efficiently used as the pressing force of pressing the sensing cuff 73 toward the wrist 200 side.

Note that when the modified example of the blood pressure measurement device 1 is attached to the wrist 200 having the relatively long circumference of the wrist 200, for example, when the blood pressure measurement device 1 is attached to the wrist 200 having the assumed longest circumference of the wrist 200, as the device body 3 indicated by the two-dot chain line in FIG. 26, the position of the blood pressure measurement device 1 in the circumferential direction of the wrist 200 may be adjusted. In this case, for example, the position in the circumferential direction of the wrist 200 of the blood pressure measurement device 1 may be adjusted so that the central portion in the circumferential direction of the sensing cuff 73 faces the radial artery 211.

The curler 5 is also applicable to the blood pressure measurement devices 1A, 1B, and 1C of the second to fourth embodiments.

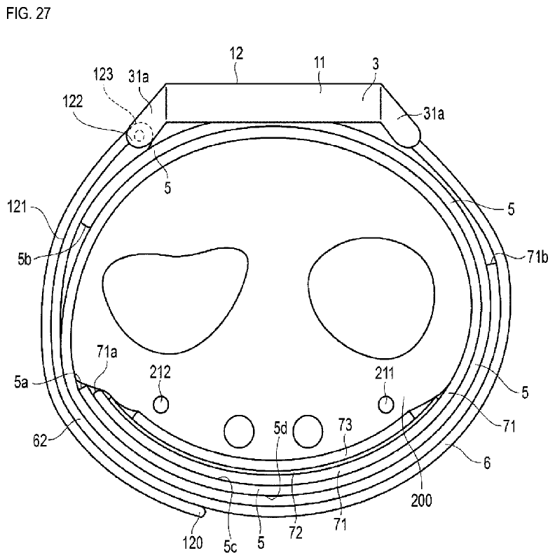

Additionally, in the example described above, the configuration in which the blood pressure measurement device 1, 1A, 1B, or 1C uses the belt 4 as a fixture has been described as an example, but the configuration is not limited thereto. As another example, the blood pressure measurement device 1, 1A, 1B, or 1C may be configured to be fixed to the wrist 200 by a band-like band having a surface fastener on the surface. An example of a configuration that applies this modified example to the blood pressure measurement device 1 of the first embodiment will be described using FIG. 27.

FIG. 27 is an explanatory diagram illustrating a state where the blood pressure measurement device 1 according to the modified example is attached to the wrist 200. The wrist 200 illustrated in FIG. 27 is the wrist 200 having the assumed shortest circumference as the circumference of the wrist 200 among the wrists 200 of the plurality of users set as targets using the blood pressure measurement device 1. The cuff structure 6 illustrated in FIG. 27 is in an inflated state.

As illustrated in FIG. 27, the blood pressure measurement device 1 according to the modified example differs from the blood pressure measurement device 1 in that a band 120 is provided instead of the belt 4. The other configurations of the blood pressure measurement device 1 according to the modified example are the same as those of the blood pressure measurement device 1.

The band 120 is formed in a band-like shape. A surface fastener 121 is provided on one surface of the band 120. A hook is provided in a region of a portion of the surface fastener 121, and a loop is provided in a region of another portion of the surface fastener 121. The region where the hook is provided and the region where the loop is provided in the surface fastener 121 are formed so as to face one another by folding the band 120 back at a folded back portion 122, which will be described later, in the wrist 200 having any circumference from the assumed shortest circumference to the assumed longest circumference of the wrist 200.

The folded back portion 122 at which the band 120 is folded is provided at a symmetrical position in the circumferential direction with respect to a position where a first end of the band 120 is fixed in the case 11. The folded back portion 122 includes, for example, a rod portion 123 extending in the width direction of the curler 5.

The band 120 is inserted between the case 11 and the rod portion 123 and folded back at the rod portion 123. The band 120 that is folded back is fixed to a portion of another portion of the band 120 with the surface fastener 121.

The band 120 configured in this manner is also applicable to the blood pressure measurement devices 1A, 1B, and 1C of the second to fourth embodiments.

In addition, in the example described above, as an example of formation of the sensing cuff 73 having the size at which the sensing cuff 73 can contact the region where the artery 210 is present in the wrist 200, the length in the longitudinal direction is set to be the length at which the sensing cuff 73 can contact the region where the radial artery 211 and the ulnar artery 212 are present in the wrist 200 having the assumed shortest circumference as the circumference of the wrist 200 and can contact the region where one of the radial artery 211 or the ulnar artery 212 is present in the wrist 200 having the assumed longest circumference as the circumference of the wrist 200. Nevertheless, the configuration is not limited to this.

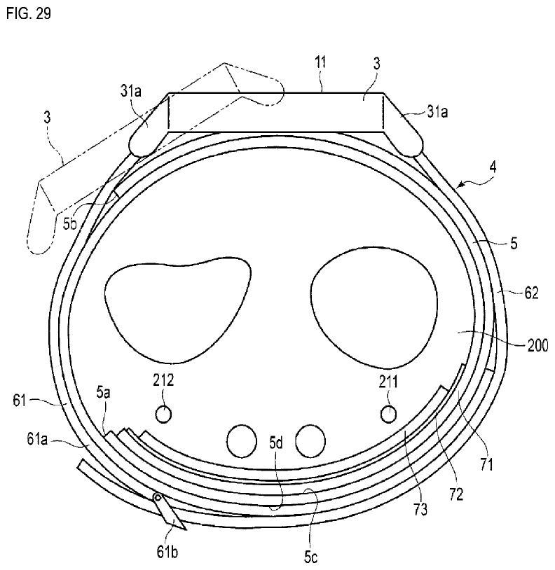

In another example, as illustrated in FIG. 29, the sensing cuff 73 may have a length at which the sensing cuff 73 can contact the region where the radial artery 211 and the ulnar artery 212 are present in the wrist 200 having the assumed longest circumference as the circumference of the wrist 200.

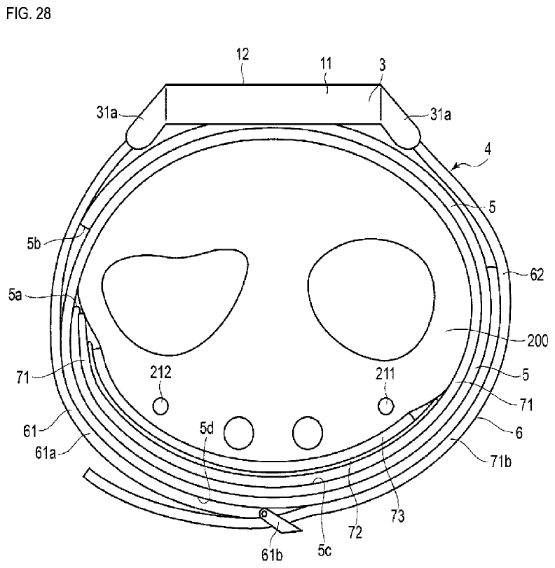

FIGS. 28 and 29 illustrate an example of application of this modified example to the blood pressure measurement device 1 of the first embodiment. FIGS. 28 and 29 are explanatory diagrams illustrating a state where the blood pressure measurement device 1 is attached to the wrist 200. FIG. 28 illustrates an example of a state where the blood pressure measurement device 1 is attached to the wrist 200 having the assumed shortest circumference of the wrist 200 among the wrists 200 of the plurality of users assumed as the targets using the blood pressure measurement device 1. The cuff structure 6 illustrated in FIG. 28 is in an inflated state. FIG. 29 illustrates an example of a state where the blood pressure measurement device 1 is attached to the wrist 200 having the assumed longest circumference of the wrist 200 among the wrists 200 of the plurality of users assumed as the targets using the blood pressure measurement device 1. The cuff structure 6 illustrated in FIG. 29 is in an inflated state.

As illustrated in FIGS. 28 and 29, the sensing cuff 73 has the length at which the sensing cuff 73 contacts the region where the radial artery 211 and the ulnar artery 212 are present in the wrist 200 having the assumed longest circumference of the wrist 200. As illustrated in FIG. 28, in a state where the blood pressure measurement device 1 is attached to the wrist 200 having the assumed shortest circumference of the wrist 200, a first end 73a of the sensing cuff 73 is disposed on the lateral side of the wrist 200 beyond the ulnar artery 212, but contacts the region where the radial artery 211 and the ulnar artery 212 are present in the wrist 200.

Note that when the modified example of the blood pressure measurement device 1 is attached to the wrist 200 having the relatively long circumference of the wrist 200, for example, when the blood pressure measurement device 1 is attached to the wrist 200 having the assumed longest circumference of the wrist 200, as the device body 3 indicated by the two-dot chain line in FIG. 29, the position of the blood pressure measurement device 1 in the circumferential direction of the wrist 200 may be adjusted. In this case, for example, the position in the circumferential direction of the wrist 200 of the blood pressure measurement device 1 may be adjusted so that the central portion in the circumferential direction of the sensing cuff 73 faces the radial artery 211.

That is, the present invention is not limited to the embodiments described above, and various modifications can be made in an implementation stage within a range that does not depart from the gist of the present invention. Furthermore, each of the embodiments may be implemented in combination as appropriate to the extent possible, and in this case, combined effects can be obtained. Also, the embodiments described above include various stages of invention, and various inventions may be extracted by appropriately combining the described plurality of disclosed constituent elements. For example, in a case where the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 Belt
5 Curler
6 Cuff structure
11 Case
12 Display unit
13 Operation unit
31 Outer case
31a Lug
31b Spring rod
32 Windshield
41 Button
61 First belt
61a Belt portion
61b Buckle
61e Frame body
61f Prong
62 Second belt
62a Small hole
71 Pressing cuff
72 Back plate
72a Groove
73 Sensing cuff
74 Tensile cuff
81 Air bag
84 Connection portion
86 Sheet member
86a First sheet member
86b Second sheet member
86b1 Opening
86c Third sheet member
86c1 Opening
86d Fourth sheet member
91 Air bag
92 Flow path body
93 Connection portion
96 Sheet member
96a Fifth sheet member
96b Sixth sheet member
101 Air bag
103 Connection portion
106 Sheet member
106a Seventh sheet member
106b Eighth sheet member
106b1 Opening
106c Ninth sheet member
106c1 Opening
106d Tenth sheet member
106d1 Opening
106e Eleventh sheet member
106e1 Opening
106f Twelfth sheet member
106f1 Opening
106g Thirteenth sheet member
106g1 Opening
106h Fourteenth sheet member
106h1 Opening
106i Fifteenth sheet member
106i1 Opening
106j Sixteenth sheet member
106j1 Opening
106k Seventeenth sheet member
106k1 Opening
106l Eighteenth sheet member
120 Band
121 Surface fastener
122 Folded back portion
123 Rod portion
130 Cuff
200 Wrist
210 Artery
211 Radial artery
212 Ulnar artery

What is claimed is:

1. A cuff structure provided in a curler of a blood pressure measurement device that includes a device body and the curler provided in the device body, the curler curving following along a circumferential direction of a wrist, the cuff structure comprising:
a sensing cuff provided on a side of an inner circumferential surface of the curler, the sensing cuff contacting a region where an artery is present in the wrist; and
a cuff having a portion provided between the curler and the sensing cuff, a portion of another portion of the cuff being disposed in a region where the sensing cuff is present in an outer circumferential surface of the curler with the blood pressure measurement device attached to the wrist, the cuff inflating to press the sensing cuff against the wrist, wherein
both ends of the curler are spaced apart, and
the portion of the other portion is configured in a shape that protrudes in the circumferential direction of the curler beyond an end of the curler.

2. The cuff structure according to claim 1, wherein
the cuff has a length such that the portion of the other portion is disposed in the region where the sensing cuff is present in the outer circumferential surface of the curler when the cuff is attached to the wrist having any circumference from an assumed shortest circumference to an assumed longest circumference of the wrist.

3. The cuff structure according to claim 1, wherein
the cuff is configured to have a shape extending from the portion to the portion of the other portion.

4. The cuff structure according to claim 1, wherein
the cuff includes a first cuff and a second cuff, the first cuff is provided between the curler and the sensing cuff, and the second cuff is provided on a back of a hand of the wrist of the curler, and
a portion of the second cuff is disposed in the region where the sensing cuff is present in the outer circumferential surface of the curler with the blood pressure measurement device attached to the wrist.

5. A blood pressure measurement device, comprising:
a device body;
a curler provided in the device body and curving following along a circumferential direction of a wrist;
a sensing cuff provided on a side of an inner circumferential surface of the curler, the sensing cuff contacting a region where an artery is present in the wrist; and
a cuff having a portion provided between the curler and the sensing cuff, a portion of another portion of the cuff being disposed in a region where the sensing cuff is present in an outer circumferential surface of the curler with the curler, the sensing cuff, and the cuff attached to the wrist, the cuff inflating to press the sensing cuff against the wrist, wherein
both ends of the curler are spaced apart, and
the portion of the other portion is configured in a shape that protrudes in the circumferential direction of the curler beyond an end of the curler.

6. The blood pressure measurement device according to claim 5, wherein
the cuff has a length such that the portion of the other portion is disposed in the region where the sensing cuff is present in the outer circumferential surface of the curler when the curler, the sensing cuff, and the cuff are attached to the wrist having any circumference from an assumed shortest circumference to an assumed longest circumference of the wrist.

7. The blood pressure measurement device according to claim 5, wherein
the cuff is configured to have a shape extending from the portion to the portion of the other portion.

8. The blood pressure measurement device according to claim 5, wherein
the cuff includes a first cuff and a second cuff, the first cuff is provided between the curler and the sensing cuff, and the second cuff is provided on a back of a hand of the wrist of the curler, and
a portion of the second cuff is disposed in the region where the sensing cuff is present in the outer circumferential surface of the curler with the curler, the sensing cuff, the first cuff, and the second cuff attached to the wrist.

9. The blood pressure measurement device according to claim 5, wherein
the curler has a first end that overlaps with a portion of another portion of the curler.
